SEQUENCE LISTING (5 sequences)

SEQ ID NO: 1 - DNA, 29 bp, synthetic construct
  source 1..29: mol_type = other DNA
aactgtgggtgggcgtggcctgaggcccc

SEQ ID NO: 2 - DNA, 128 bp, synthetic construct
  source 1..128: mol_type = other DNA
tagttagtaaactctttgaccctacatctctacagatataaatgagtgtgagaccacaaacgagtgccgggaggatgaaatgtgttggaattatcatggcggcttccgttgttatccacgaaatcctt

SEQ ID NO: 3 - DNA, 139 bp, Homo sapiens
  source 1..139: mol_type = genomic DNA
agtttagttagtaaactctttgaccctacatctctacagatataaatgagtgtgagaccacaaatgaatgctgggaggatgaaatgtgttggaattatcatggcggcttccgttgttatccacgaaatccttgtcaaga

SEQ ID NO: 4 - protein, 33 aa, Homo sapiens
DINECETTNECWEDEMCWNYHGGFRCYPRNPCQ

SEQ ID NO: 5 - DNA, 500 bp, Homo sapiens
  source 1..500: mol_type = genomic DNA
aagctatgaaaacacaagccctgggagctgagatatgtcctaacttacccagctgagctgtgaggtgtgagtggctctaacattttccagttgtttctgaggacctcagatcaaagcttccctttgcctaaaagcatctgcctgctggtgtgggcccttgggggccgtcacagtgcactgaggttagagtcctgcaagggtgaacccttatgtaacaagtagttgggcaagtttacagctctctgtaatctgacagtagagtccagactggtttgatgaaagagggtaaactgtgggtgggcgtggcctgaggccccactagaagcccagggagatctgaggaaagggagggcttttctgatctctcccaattagaggattaggcaattggcagcgcagggcggtaactctgggcggggctgggctccagggctggacagcacagtccctctgaactgcacagagacctcgcaggccccgagaactgtcgcccttccacg

(19) United States
(12) Patent Application Publication
(10) Pub. No.: US 2024/0050590 A1
(43) Pub. Date: Feb. 15, 2024

(54) METHODS AND COMPOSITIONS TO REGULATE CHOLESTEROL EFFLUX TO PREVENT, TREAT, OR CURE MACULAR DEGENERATION

(71) Applicant: The Trustees of Columbia University in the City of New York, New York, NY (US)

(72) Inventors: Yi-Ting Tsai, New York, NY (US); Stephen H. Tsang, New York, NY (US)

(21) Appl. No.: 18/495,538

(22) Filed: Oct. 26, 2023

Related U.S. Application Data
(63) Continuation of application No. PCT/US2022/026508, filed on Apr. 27, 2022.
(60) Provisional application No. 63/180,201, filed on Apr. 27, 2021.

(51) Int. Cl. A61K 48/00; C12N 15/86; A61K 38/18

(52) U.S. Cl. CPC A61K 48/005; C12N 15/86; A61K 38/1808; C12N 2750/14143

(57) ABSTRACT

The present disclosure relates to methods for regulating cholesterol efflux in retinal pigment epithelium cells and treating or preventing a neurodegenerative disease or disorder (e.g., macular degeneration).

### CROSS-REFERENCE TO RELATED APPLICATIONS

This application is a continuation of International Application No. PCT/US2022/026508, filed Apr. 27, 2022, which claims the benefit of U.S. Provisional Application No. 63/180,201, filed Apr. 27, 2021, the contents of which are herein incorporated by reference in its entirety.

### STATEMENT REGARDING FEDERALLY SPONSORED RESEARCH OR DEVELOPMENT

This invention was made with government support under 5P30CA013696, U54OD020351, R24EY028758, R24EY027285, 5P30EY019007, R01EY018213, R01EY024698, R01EY024091, R01EY026682, U01EY030580, and R21AG050437 awarded by the National Institutes of Health. The government has certain rights in the invention.

### TECHNICAL FIELD

The present disclosure relates to methods for regulating cholesterol efflux in retinal pigment epithelium cells and treating or preventing a neurodegenerative disease or disorder (e.g., macular degeneration).

### SEQUENCE LISTING STATEMENT

The contents of the computer readable sequence listing filed herewith, titled “COLUM-40033.302.xml”, created Oct. 26, 2023, having a file size of 6,222 bytes, is hereby incorporated by reference in its entirety.

### BACKGROUND OF THE INVENTION

Macular degeneration (MD) is a heterogenous group of severe neurodegenerative diseases characterized by retinal pigment epithelium (RPE) dysfunction, leading to progressive vision loss. Affecting more than 170 million individuals, MD is a leading cause of visual disabilities worldwide. Age is a risk factor for MD, and as the human lifespan increases, age-related macular degeneration will be a growing public health concern. By 2040, more than 288 million people are expected to be diagnosed with MD, outpacing all invasive cancers combined and more than double the prevalence of Alzheimer disease (MIM: 104300).

Early stages of MD can be identified on fundus imaging by the presence of yellow spots called drusen, which correspond to deposits of excess lipids and proteins between the basal lamina of the RPE and Bruch's membrane. Drusen are thought to cause vision loss through geographic atrophy and choroidal neovascularization. Studies exploring the mechanism of deposit formation have suggested the involvement of complement risk factors and the complement system. Nonetheless, efforts to further elucidate the pathophysiology of disease and develop effective treatment strategies have fallen short as the vast majority of these studies have focused primarily on AMD. While by far the most prevalent and therefore devastating of all the different forms of MD, AMD as a disease model for MD is limited due to the variability in its genetic and environmental causes.

### SUMMARY OF THE INVENTION

Provided herein are methods for delaying the onset of, treating, preventing and/or curing a neurodegenerative disease in a subject in need thereof. In some embodiments, the neurodegenerative disease comprises macular degeneration.

In some embodiments, the methods comprise administering to a subject an effective amount of one or more agents which increase the expression or activity of carboxylesterase 1 (CES1) in retinal pigment epithelial cells (RPE).

In some embodiments, the one or more agents comprises one or more nucleic acids encoding CES1, or an active fragment or variant thereof. In some embodiments, the one or more nucleic acids comprises one or more viral vectors which target the RPE. In some embodiments, the one or more viral vectors comprise an adeno-associated viral (AAV) vector. In some embodiments, the one or more viral vectors further comprise an RPE specific promoter.

In some embodiments, the one or more agents comprise one or more epidermal growth factor receptor (EGFR) signaling pathway agonists, or nucleic acids encoding thereof. In some embodiments, the one or more EGFR signaling pathway agonists comprise epidermal growth factor (EGF).

In some embodiments, the one or more agents comprises one or more inhibitors of SP1.

In some embodiments, the one or more agents regulate cholesterol efflux in retinal pigment epithelial cells (RPE).

In some embodiments, the methods further comprise instructing the subject to ingest a low fat diet.

In some embodiments, the methods comprise administering to the subject an effective amount of one or more nucleic acids encoding a gene product which regulates cholesterol efflux.

In some embodiments, the nucleic acids are configured to express the gene product in retinal pigment epithelial cells (RPE). In some embodiments, the gene product comprises CES1 or an active fragment or variant thereof. In some embodiments, the one or more nucleic acids comprises one or more viral vectors which target the RPE. In some embodiments, the one or more viral vectors comprise an adeno-associated viral (AAV) vector. In some embodiments, the one or more viral vectors further comprise an RPE specific promoter.

Other aspects and embodiments of the disclosure will be apparent in light of the following detailed description and accompanying figures.

### BRIEF DESCRIPTION OF THE DRAWINGS

FIGS. 1A-1H show gene correction of EFEMP1^{R345W i}n iPSC from individuals with DHRD FIG. 1A is a flowchart of the preparation of iRPEs used. FIG. 1B is the design of gRNA and donor template (SEQ ID NO: 2) used for the gene correction of SEQ ID NO: 3. The expected cutting site is indicated by the red arrow. The p.Arg345Trp mutation is marked by red box in a fragment of EFEMP1 (SEQ ID NO: 4). The gRNA protospacer is marked in cyan while the donor template is outlined in yellow. The donor template contains two silent mutations (N342 and E343) in addition to the R345 mutation to enable screening by RFLP. FIG. 1C are images of Z0.1 (red) immunostaining of iRPE with DAPI counterstaining (blue). FIG. 1D are images of the immunostaining results of BEST1. FIG. 1E are images of the immunostaining results of RPE65. FIG. 1F is representative western blot result of RPE65 expression in iRPE lysates. FIG. 1G is representative western blot result of CRALBP expression in iRPE lysates. FIG. 1H is representative western blot result of EFEMP1 expression in iRPE lysates and culture supernatants. Scale bar=100 μm.

FIGS. 2A-2G show the proteomic analysis of iPRE cells. FIG. 1A is a 10×10 dot plot presenting the percentage of differentially expressed proteins (DEPs) after gene correction. The criteria defining DEP: >2-fold difference, p<0.001. FIG. 2B is a circos plot presenting the biological process attributes of the DEPs shown in FIG. 2A. The colored boxes next to the gene labels indicate the change in expression after gene correction (red: increase; blue: decrease). FIG. 2C is a Venn diagram of the DEPs. FIGS. 2D and 2E are volcano plots presenting the DEPs in different comparisons: FIG. 2D—EFEMP1^{R345W} (n=6) versus EFEMP1^{WT} (n=6); FIGS. 2E —EFEMP1^{corrected} (n=12) versus EFEMP1^{R345W} (n=6). FIG. 2F is representative western blot result of CES1 expression of iRPE culture. FIG. 2G is representative immunocytochemistry result of CES1 staining in iRPE culture. Scale bar=100 μm.

FIGS. 3A-3P show intracellular lipid accumulation by EFEMP1^{R345W} via reducing cholesterol efflux. FIG. 3A is representative CES1 staining of human retina section. A positive staining signal was detected in the RPE cells (yellow arrow), outer segment of photoreceptor (green arrow), outer plexiform layer (cyan arrows), and inner limiting membrane (purple arrows). Scale bar=50 μm (n=3). FIG. 3B is Nile red staining of iRPE cells. Scale bar=100 FIGS. 3C and 3D are graphs of the amount (FIG. 3C) and size (FIG. 3D) of the lipid droplet detected in FIG. 3B (n=7). FIG. 3E is a graph of the cholesterol efflux of iRPE cells shown in FIG. 3B (n=2). FIG. 3F is Nile red staining of EFEMP1^{WT} iRPE cells after CES1 knockdown. Scale bar=100 FIGS. 3G and 3H are graph of the amount (FIG. 3G) and size (FIG. 3H) of the lipid droplet detected in FIG. 3F (n=2). FIG. 3I is a graph of the cholesterol efflux of iRPE cells shown in FIG. 3F (n=2). FIGS. 3J and 3K are graphs of the amount (FIG. 3J) and size (FIG. 3K) of the lipid droplets detected in the EFEMP1^{R345W} iRPE with or without CMV-CES1 overexpression (n=2). FIG. 3L is Nile red staining of EFEMP1^{WT} iRPE cells overexpressing either EFEMP1^{WT} or EFEMP1^{R345W}. Scale bar=100 FIGS. 3M and 3N are graphs of the amount (FIG. 3M) and size (FIG. 3N) of the lipid droplet detected in FIG. 3L (n=2). FIG. 3O is a graph of the cholesterol efflux of iRPE cells shown in FIG. 3L (n=2). FIG. 3P is a graph of the relative expression levels of cholesterol efflux-related genes in EFEMP1^{WT}, EFEMP1^{R345W}, and EFEMP1^{corrected} iRPE cells. Data in FIGS. 3C-3E, 3G-3K, and 3M-3O is represented as mean±SD. Data in FIG. 3P is represented as geometric mean±SD. *p<0.05; **p<0.01; ***p<0.001.

FIGS. 4A-4D show EFEMP1^{R345W} regulates CES1 expression through EGFR signaling. FIG. 4A is representative western blot result of CES1 expression levels in normal iRPE cells after 72 h of treatment with different agonists and antagonists (n=2). FIG. 4B is representative western blot result of CES1 expression of EFEMP1^{WT}, EFEMP1^{R345W}, and EFEMP1^{corrected} iRPE cells (lane 1-3) and EFEMP1^{R345W} cells pre-treated with 100 nM EGF for 12 h (lane 4) (n=2). FIG. 4C is a schematic of the sample preparation flowchart for phosphor-array analysis. FIG. 4D is a heatmap of phosphorylation status of the EGFR downstream targets measured by phosphor-array (n=6). Data represented as mean±SD.

FIGS. 5A-5E show that EFEMP1^{R345W} perturbs CES1 expression via SP1. FIG. 5A shows the predicted transcription factor binding sites located within 500 bp upstream of the CES1 transcription start site in SEQ ID NO: 5. FIG. 5B is a graph of the relative activity of transcription factors in iRPE cells. Data represented as EFEMP1^{R345W} group/EFEMP1^{WT} group. Data represented as mean±SD (n=2). FIG. 5C shows an electrophoretic mobility shift assay of nuclear extract from EFEMP1^{WT} iRPE cells. FIG. 5D shows the results of a ChIP-qPCR performed to detect the binding of SP1 to CES1 promoter. Data represented as geometric mean±SD (n=2). FIG. 5E shows the results of a qPCR result of CES1 expression level in iRPE cells after treatment of SP1-specific inhibitor mithracin. Data represented as geometric mean±SD (n=2). *=p<0.05; **=p<0.01.

FIGS. 6A-6C show the characterization of iPSC cells. FIG. 6A is karyotyping result of the EFEMP1^{WT}, EFEMP1^{R345W} and EFEMP1^{corrected} iPSCs. FIG. 6B is images from immunostaining of stem cells markers TRA-1-60 and OCT4. FIG. 6C is images from immunostaining of stem cells marker NANOG.

FIG. 7 is the proteomic analysis of iRPE cells. Expression level of the 37 DEPs shown in FIG. 2 as determined by mass spectrometry. The order of the proteins is ranked by the mean expression level of the diseased group. EFEMP1^{WT} (n=6), EFEMP1^{R345W} (n=6), and EFEMP1^{corrected} clones (n=6).

FIG. 8 shows the expression of CES1 in two healthy donor and two DHRD patient iRPE cells. The cell lysates from iRPE derived from healthy donor #2, #3 and DHRD patient #2, #3 were determined by western blot.

FIGS. 9A-9F show the absence of cytokine changes and UPR in DHRD patient-derived iRPE cells. IL1 (FIG. 9A), IL-6 (FIG. 9B), IL-18 (FIG. 9C), TNF-alpha (FIG. 9D), IFN-alpha (FIG. 9E) and TNF-beta (FIG. 9F) cytokine level in the supernatant of EFEMP1^{WT} (n=12), EFEMP1^{R345W (}n=12), and EFEMP1^{corrected} (n=16) iRPE cell cultures. One-way ANOVA analysis showed no significant differences between the groups. Note: The scale range of each graph was set according to the range of the standard provided in the respective ELISA kits by the manufacturer. FIG. 9G is the real-time PCR analysis of UPR-related gene expression level in EFEMP1^{WT} (n=5), EFEMP1^{R345W} (n=6), and EFEMP1^{Corrected} (n=8)) iRPE cells. Geometric mean and standard deviation were used to determine the p-values. Data represented as geometric mean±geometric SD. One-way ANOVA analysis showed no significant differences between the groups.

FIG. 10 is the immunocytochemistry of UPR markers. The staining of HSPA5 (left column) and DDIT3 (middle column) of the EFEMP1^{WT}, EFEMP1^{R345}, and EFEMP1^{corrected} iRPE.

FIG. 11 is a graph of the relative expression of CES1 in retina. The CES1 mRNA level in of retina tissue derived from human autopsy and human iPSC-derived retinal organoid were determined by qPCR. The data is presented as the ratio of the level in retina/the level in RPE. Data represented as geometric mean±geometric SD. (n=2)

FIGS. 12-12C show that EGF treatment ameliorates intracellular lipid droplet accumulation and improve cholesterol efflux in EFEMP1^{R345W} iRPE. FIGS. 12A and 12B are graphs of the amount (FIG. 12A) and size (FIG. 12B) of the lipid droplet detected in EFEMP1^{R345W} iRPE with or without the treatment of 100 mM EGF for two weeks. FIG. 12C is a graph of the cholesterol efflux of EGF-treated or untreated EFEMP1^{R345W} iRPE. Data represented as mean±SD. (n=2)

FIG. 13 shows the overexpression of EFEMP1 in HEK293 cells. The HEK293 cells were transfected with pcDNA3.1 vector expressing either EFEMP1^{WT} or EFEMP1^{R345W} for 96 hours. The expression of EFEMP1 in cell lysate or culture supernatant was verified by western blot. HEK293 cell without plasmid transfection was used as a negative control.

### DETAILED DESCRIPTION OF THE INVENTION

The present disclosure provides methods for regulating cholesterol efflux and treating or preventing macular degeneration.

Macular degeneration (MD) is characterized by the progressive deterioration of the macula and represents one of the most prevalent causes of blindness worldwide. Abnormal intracellular accumulation of lipid droplets and pericellular deposits of lipid-rich material in the retinal pigment epithelium (RPE) called drusen are clinical hallmarks of different forms of MD including Doyne honeycomb retinal dystrophy (DHRD) and age-related MD (AMD). However, the appropriate molecular therapeutic target underlying these disorder phenotypes remains elusive. Herein the proteomic profiles of induced pluripotent stem cell (iPSC)-derived RPEs (iRPE) from individuals with DHRD and their isogenic controls were compared. The analysis and follow-up studies elucidated the mechanism of lipid accumulation in DHRD iRPE cells. Specifically, significant downregulation of carboxylesterase 1 (CES1), an enzyme that converts cholesteryl ester to free cholesterol, an indispensable process in cholesterol export was detected. CES1 knockdown or overexpression of EFEMP1^{R345W}, a variant of EGF-containing fibulin extracellular matrix protein 1 that is associated with DHRD and attenuated cholesterol efflux and led to lipid droplet accumulation. In iRPE cells, EFEMP1^{R345W} had a hyper-inhibitory effect on epidermal growth factor receptor (EGFR) signaling when compared to EFEMP1^{WT} and appeared to suppress CES1 expression via the downregulation of transcription factor SP1. Taken together, these results highlighted the homeostatic role of cholesterol efflux in iRPE cells and identified CES1 as a mediator of cholesterol efflux in MD.

This study was focused on a form of MD with a monogenic etiology: Doyne honeycomb retinal dystrophy (DHRD[MIM:126600]). DHRD is a rare inherited macular dystrophy that causes irreversible central vision loss later in life due to geographic atrophy and choroidal neovascularization. Like other forms of MD, an early indication of DHRD is the development of drusen, which pattern the fundus in a honeycomb-like fashion. Although there is variability in disease progression, DHRD-affected individuals exhibit classical MD findings of RPE hypertrophy and abnormal subretinal fibrosis.

To date, the only identified causative gene for DHRD is EFEMP1 (MIM:601548), which encodes epidermal growth factor (EGF)-containing fibulin-like extracellular matrix protein 1, also known as fibulin-3 (F3). EFEMP1 is one of eight glycoproteins in the fibulin family of extracellular matrix (ECM) glycoproteins. All proteins in this family contain a series of calcium-binding EGF domains followed by a C-terminal fibulin-type domain. These fibulin proteins are secreted and integrated into the ECM, where they play a critical role in basement membrane formation. Strikingly, mutations in three of the eight fibulin proteins—F3, F5, and F6—have been found or are suspected to contribute to the development of AMD or related retinal degeneration.

DHRD is caused by a single heterozygous missense mutation (p.Arg345Trp [c.1033C>T]) in EFEMP1. The autosomal-dominant inheritance pattern suggests a toxic gain-of-function mechanism. Previous studies have generated different mouse models of DHRD with various levels of success. Both Efemp1^{R345W+} single dominant mice and knockout mice expressing no Efemp1 have no observable problems in the eye. On the other hand, Efemp1^{R345W/R345W} double dominant mice develop pathological phenotypes in the retina including progressive development of drusen and RPE atrophy but do not genocopy individuals with DHRD.

Cell culture studies have highlighted that both AMD and DHRD lead to significant EFEMP1 immunoreactivity around the RPE in the presence of drusen. To account for this observation, studies have suggested that the p.Arg345Trp mutation prevents the proper folding and secretion of the EFEMP1 protein, activating an unfolded protein response (UPR) that triggers MD. Subsequent studies have failed to validate this model, however, and have alternatively attributed the retinal degeneration to the complement pathway. As evident from this lack of consensus, the question of how the p.Arg345Trp mutation in EFEMP1 causes drusen formation in DHRD remains unanswered; as such, further investigation of the role of EFEMP1 in the retina is warranted.

In addition to EFEMP1^{R345W/R345W} and EFEMP1^{-/-} mouse models, previous studies have also characterized the EFEMP1 p.Arg345Trp variant by overexpressing EFEMP1^{R345W} in ARPE-19 cells, a human retinal pigment epithelial cell line. These systems, however, are limited in their capacity as disease models for DHRD as they are not translatable to humans. At the same time, heterozygous knock-in of the R345W EFEMP1 mutation in mice results in insignificant phenotypes. To overcome these problems, human induced pluripotent stem cells (iPSC)-derived RPE (iRPE) cells were used to study the pathogenesis of drusen formation in DHRD.

Section headings as used in this section and the entire disclosure herein are merely for organizational purposes and are not intended to be limiting.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of, molecular biology, immunology, and protein and nucleic acid chemistry described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, the terms “administering,” “providing,” and “introducing,” are used interchangeably herein and refer to the placement into a subject by a method or route which results in at least partial localization a desired site. Administration can be by any appropriate route which results in delivery to a desired location in the subject.

An “effective amount” refers to an amount sufficient to elicit a desired biological response (e.g., treating a condition). As will be appreciated by those skilled in the art, the effective amount may vary depending on such factors as the desired biological endpoint, pharmacokinetics, the condition being treated, the mode of administration, and the age and health of the subject. An effective amount encompasses therapeutic and prophylactic treatment. A “therapeutically effective amount” is an amount sufficient to provide a therapeutic benefit in the treatment of a condition, or to delay or minimize one or more symptoms associated with the condition. In some embodiments, a therapeutically effective amount is an amount sufficient to provide a therapeutic benefit in the treatment of a condition or to minimize one or more symptoms associated with the condition. A therapeutically effective amount means an amount, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

The term “gene” refers to a DNA sequence that comprises control and coding sequences necessary for the production of an RNA having a non-coding function (e.g., a ribosomal or transfer RNA), a polypeptide, or a precursor of any of the foregoing. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or an RNA chain that has functional role to play in an organism. For the purpose of this disclosure it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The term “gene product,” as used herein, refers to any biochemical product resulting from expression of a gene. Gene products may be RNA or protein. RNA gene products include non-coding RNA, such as tRNA, rRNA, micro RNA (miRNA), and small interfering RNA (siRNA), and coding RNA, such as messenger RNA (mRNA).

As used herein, a “nucleic acid” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. The term “nucleic acid” may also encompass a chain comprising non-natural nucleotides, modified nucleotides, and/or non-nucleotide building blocks that can exhibit the same function as natural nucleotides (e.g., “nucleotide analogs”). Further, the term “nucleic acid” as used herein refers to an oligonucleotide, nucleotide or polynucleotide, and fragments or portions thereof, and to DNA or RNA of genomic or synthetic origin, which may be single or double-stranded, and represent the sense or anti sense strand.

As used herein, the term “preventing” refers to partially or completely delaying onset of a disease, disorder and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition.

As used herein, “treat,” “treating,” and the like means a slowing, stopping, or reversing of progression of a disease or disorder. The term also means a reversing of the progression of such a disease or disorder. As such, “treating” means an application or administration of the methods or devices described herein to a subject, where the subject has a disease or a symptom of a disease, where the purpose is to cure, heal, alleviate, relieve, alter, remedy, ameliorate, improve, or affect the disease or symptoms of the disease.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of devices and systems contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods herein, the mammal is a human.

A “vector” or “expression vector” is a replicon, such as plasmid, phage, virus, or cosmid, to which another DNA segment, e.g., an “insert,” may be attached or incorporated so as to bring about the replication of the attached segment in a cell.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified,” “mutant,” or “polymorphic” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. METHODS

The present disclosure provides methods for delaying the onset of, treating, preventing and/or curing a neurodegenerative disease in a subject.

In some embodiments, the neurodegenerative disease is an ocular disease. In some embodiments, the ocular disease is characterized by accumulation of drusen, lipid droplets and pericellular deposits of lipid-rich material, in the retinal pigment epithelium (RPE) or under the retina. In some embodiments, the neurodegenerative disease is macular degeneration. The macular degeneration may include Doyne honeycomb retinal dystrophy (DHRD), also known as Malattia Leventinese or Familial Dominant Drusen, and age-related MD (AMD). The macular degeneration may be “dry” (atrophic) type or “wet” (exudative) type. In some embodiments, the macular degeneration is “dry” (atrophic) type. The macular degeneration may be early stage, intermediate stage, or late stage macular degeneration.

In some embodiments, the methods comprise administering to the subject an effective amount of one or more agents which increase the expression or activity of CES1 in retinal pigment epithelium (RPE) cells.

In some embodiments, the one or more agents comprise one or more nucleic acids encoding CES1, or an active fragment or variant thereof. In some embodiments, the CES1 is wild-type CES1. In some embodiments, the CES1 is an active fragment or variant thereof of wild-type CES1, such that the primary amino acid sequence may contain one or more substitutions or truncations compared to wild-type while the resulting polypeptide retains its expression levels, cellular localization and/or activities.

An amino acid “replacement” or “substitution” refers to the replacement of one amino acid at a given position or residue by another amino acid at the same position or residue within a polypeptide sequence. Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or He), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gin), lysine (K or Lys), and arginine (R or Arg).

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra).

Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH2 can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

In some embodiments, the one or more agents comprise one or more epidermal growth factor receptor (EGFR) signaling pathway agonists, or one or more nucleic acids encoding thereof. EGFR is an intrinsic membrane protein composed of an extracellular ligand-binding domain connected to an intracellular tyrosine kinase domain by a single transmembrane α-helix. Upon binding an agonist, the EGF receptor dimerizes leading to the activation of its tyrosine kinase and the phosphorylation of tyrosine residues in the C-terminal tail of the receptor. The phosphorylated tyrosines on the EGF receptor serve as binding sites for a large number of signaling proteins that contain SH2 and/or phosphotyrosine-binding domains. Some of these proteins, such as Cb1, possess an enzymatic activity. Others, such as Grb2 or Shc, serve as adapter proteins that bring other proteins into the EGFR-containing complex. For example, Grb2 recruits the scaffolding protein, Gab 1, to EGFR. Phosphorylation of Gab 1 by the EGFR allows Gab 1 to recruit additional proteins, such as Shp2 or PI3K, to the signaling complex. The recruitment of these signaling proteins to the receptor ultimately triggers the activation of a variety of downstream signaling pathways, thereby mediating the intracellular effects of growth factor binding.

The one or more epidermal growth factor receptor (EGFR) signaling pathway agonists may act directly on EGFR or any of the members of the downstream signaling pathways (e.g., the Ras/Raf signaling cascade, phosphatidylinositol 3-kinase/AKT signaling cascade, Signal Transducers and Activators of Transcription (STAT) pathway). The agonists may include endogenous agonists or engineered variants thereof. In some embodiments, the agonists are small molecules. In some embodiments, the agonists are peptides, polypeptides, proteins, nucleic acids, and the like.

In some embodiments, the one or more epidermal growth factor receptor (EGFR) signaling pathway agonists may act directly on EGFR. EGFR can bind a number of different agonist ligands including high affinity ligands (e.g., EGF, TGFα, BTC, and HB-EGF) and low affinity ligands (AREG, EPG, and EPR). In select embodiments, the one or more EGFR signaling pathway agonists comprise epidermal growth factor (EGF).

In some embodiments, the one or more EGFR signaling pathway agonists act on members of the downstream signaling pathways. In some embodiments, the one or more EGFR signaling pathway agonists activate the PI3K/AKT signaling pathway.

In some embodiments, the one or more agents comprise one or more inhibitors of transcription factor Sp1, also known as specificity protein 1. The inhibitors may comprise small molecule inhibitors, interfering RNAs (e.g., microRNA (miRNA), small hairpin RNA (shRNAs), double stranded RNA (dsRNA) and small interfering RNA (siRNA)), or antibodies. As used herein, the term “small molecules” encompasses molecules other than proteins or nucleic acids without strict regard to size. Non-limiting examples of small molecules that may be used according to the methods and compositions of the present invention include, small organic molecules, peptide-like molecules, peptidomimetics, carbohydrates, lipids or other organic (carbon containing) or inorganic molecules. In some embodiments, the inhibitor is selected from: mithramycin and analogs thereof, and plicamysin. In some embodiments, the inhibitor is an interfering RNA.

In some embodiments, the one or more agents regulate cholesterol efflux in RPE cells. In some embodiments, the methods comprise administering to the subject an effective amount of one or more nucleic acids encoding a gene product which regulates cholesterol efflux. In some embodiments, the gene product comprises carboxylesterase 1 (CES1) or an active fragment or variant thereof.

Nucleic acids of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Moreover, inducible expression can be accomplished by placing the nucleic acid encoding such a molecule under the control of an inducible promoter/regulatory sequence. Promoters that are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

In some embodiments, the nucleic acids are configured for expression in the RPE. In some embodiments, the nucleic acids comprise an RPE specific promoter. RPE specific promoters include, but are not limited to, vitelliform macular dystrophy (VMD2) promoter, bestrophin-1 (BEST1) promoter, RPE65 promoter, or a synthetic RPE promoter (see, for example, Johari et al., Biotechnol Bioeng. 2021 May; 118(5):2001-2015).

The present disclosure also provides for vectors containing the nucleic acids and cells containing the nucleic acids or vectors, thereof. The vectors may be used to propagate the nucleic acid in an appropriate cell and/or to allow expression from the nucleic acid (e.g., an expression vector). The person of ordinary skill in the art would be aware of the various vectors available for propagation and expression of a nucleic acid sequence.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

The vectors of the present disclosure may direct the expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining. In some embodiments, the vectors direct the expression of the nucleic acid in the RPE.

Additionally, the vector may contain, for example, some or all of the following: a selectable marker gene for selection of stable or transient transfectants in host cells; transcription termination and RNA processing signals; 5′-and 3′-untranslated regions; internal ribosome binding sites (IRESes), versatile multiple cloning sites; and reporter gene for assessing expression of the chimeric receptor. Suitable vectors and methods for producing vectors containing transgenes are well known and available in the art. Selectable markers include chloramphenicol resistance, tetracycline resistance, spectinomycin resistance, neomycin, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRP1 genes of S. cerevisiae.

The vectors may be maintained as an autonomously replicating sequence or extrachromosomal element or may be integrated into host DNA.

Viral and non-viral based gene transfer methods can be used to introduce the nucleic acids into cells, tissues, or a subject. Such methods can be used to administer the nucleic acids to cells in culture, or in a host organism. Non-viral vector delivery systems include DNA plasmids, cosmids, RNA (e.g., a transcript of a vector described herein), a nucleic acid, and a nucleic acid complexed with a delivery vehicle.

Viral vector delivery systems include DNA and RNA viruses, which have either episomal or integrated genomes after delivery to the cell. A variety of viral constructs may be used to deliver the present nucleic acids to the cells, tissues and/or a subject. Viral vectors include, for example, retroviral, lentiviral, adenoviral, adeno-associated, baculoviral, and herpes simplex viral vectors. Nonlimiting examples of such recombinant viruses include recombinant adeno-associated virus (AAV), recombinant adenoviruses, recombinant lentiviruses, recombinant retroviruses, recombinant herpes simplex viruses, recombinant baculoviruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7(1):33-40; and Walther W. and Stein U., 2000 Drugs, 60(2): 249-71, incorporated herein by reference.

In some embodiments, the one or more nucleic acids comprises one or more viral vectors which target the RPE. In some embodiments, the one or more viral vectors comprise an adeno-associated viral (AAV) vector. In some embodiments, the one or more viral vectors further comprise an RPE specific promoter, as described above. As such, the disclosure also provides recombinant AAVs encoding on or more of the disclosed nucleic acids.

Methods of delivering vectors to cells are well known in the art and may include DNA or RNA electroporation, transfection reagents such as liposomes, microspheres, or nanoparticles to delivery DNA or RNA; delivery of DNA, RNA, or protein by mechanical deformation (see, e.g., Sharei et al. Proc. Natl. Acad. Sci. USA (2013) 110(6): 2082-2087, incorporated herein by reference); or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the construct containing the one or more nucleic acids encoding CES1, gene product which regulates cholesterol efflux, or the one or more epidermal growth factor receptor signaling pathway agonists can be delivered by any method appropriate for introducing nucleic acids into a cell.

In the methods disclosed herein, administration may be by any of those methods known in the art that facilitate administration systemically/peripherally or at the site of desired action, including but not limited to, oral (e.g., by ingestion); topical (including e.g. transdermal, intranasal, ocular, buccal, and sublingual); pulmonary (e.g., by inhalation or insufflation therapy using, e.g., an aerosol, e.g., through mouth or nose); rectal; vaginal; parenteral (e.g., by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal injection); or by implant of a depot, for example, subcutaneously or intramuscularly. In some embodiments, the administration is topical, local ocular (e.g., subconjunctival, retrobulbar, intracameral, intravitreal), periocular (sub-conjunctival, sub-Tenon's, posterior juxtascleral, peribulbar and retrobulbar injections), suprachoroidal, sub-retinal, or systemic.

The one or more agents which increase the expression or activity of carboxylesterase 1 (CES1) (e.g., one or more nucleic acids encoding CES1, or an active fragment or variant thereof, the one or more epidermal growth factor receptor (EGFR) signaling pathway agonists, or one or more nucleic acids encoding thereof, and the one or more agents comprises one or more inhibitors of SP1) may be administered alone or in combination with each other. Combinations may be administered either concomitantly, e.g., as an admixture, separately but simultaneously or concurrently, or sequentially. This includes presentations in which the combined agents are administered together as a therapeutic mixture, and also procedures in which the combined agents are administered separately but simultaneously, e.g., as through separate intravenous lines into the same individual. Administration in combination also includes the separate administration of one agents given first, followed by administration of a second agent. Thus, combination treatments may be achieved by administering a pharmaceutical composition that includes both agents, or by administering two pharmaceutical compositions, at the same time or within a short time period.

As such, the disclosure also provides pharmaceutical compositions comprising the one or more agents which increase the expression or activity of CES1 (e.g., one or more nucleic acids encoding CES1, or an active fragment or variant thereof, the one or more epidermal growth factor receptor (EGFR) signaling pathway agonists, or one or more nucleic acids encoding thereof, and the one or more agents comprises one or more inhibitors of SP1). In some embodiments, the pharmaceutical compositions comprise recombinant AAVs comprising one or more nucleic acids (e.g., viral vectors) as described herein.

The pharmaceutical compositions and formulations may include pharmaceutically acceptable carriers. The term “pharmaceutically acceptable carrier,” as used herein, means a non-toxic, inert solid, semi-solid or liquid filler, diluent, encapsulating material, surfactant, cyclodextrins or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; surfactants such as, but not limited to, cremophor EL, cremophor RH 60, Solutol HS 15 and polysorbate 80; cyclodextrins such as, but not limited to, alpha-CD, beta-CD, gamma-CD, HP-beta-CD, SBE-beta-CD; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The route by which the disclosed agents are administered and the form of the composition will dictate the type of carrier to be used.

In some embodiments, the methods further comprise instructing the subject to ingest a low fat diet. In general, a low fat diet refers to a diet that provides between about 10% to less than about 40% (e.g., about 10%, about 15%, about 20%, about 25%, about 30%, or about 35%) of total calories from fat or a diet that is between about 10 and about 60 (e.g., about 10, about 15, about 20, about 25, about 30, about 35, about 40, about 45, about 50, or about 55) grams of fat per day.

### 3. SYSTEMS OR KITS

The present invention is also directed to systems or kits comprising one or more agents which increase the expression or activity of CES1 and/or one or more nucleic acids encoding a gene product which regulates cholesterol efflux. Descriptions provided for the one or more agents which increase the expression or activity of CES1 and/or one or more nucleic acids encoding a gene product which regulates cholesterol efflux are applicable to the disclosed systems and kits.

The kits can also comprise instructions for using the components of the kit. The instructions are relevant materials or methodologies pertaining to the kit. The materials may include any combination of the following: background information, list of components, brief or detailed protocols for using the compositions, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods. The kit may include instructions for use in any of the methods described herein.

The kits provided herein are in suitable packaging. Suitable packaging includes, but is not limited to, vials, bottles, jars, flexible packaging, and the like.

The present disclosure also provides for kits for performing the methods or producing the components in vitro. Optional components of the kit include one or more of the following: buffers, cell culture media or components thereof for generating the nucleic acids, viral vectors, and/or viruses as disclosed herein.

### 4. EXAMPLES

### Materials and Methods

iPSC culture and the differentiation of iRPE Fibroblasts from three white individuals with DHRD (aged 37, 47, and 59 years) and three healthy donors (aged 14, 55, and 64 years) were reprogrammed into iPSCs using the CytoTune-iPS 2.0 Sendai Reprogramming Kit (Thermo Fisher Scientific). The generated iPSCs were tested for their quality and pluripotency by stem cell marker staining and karyotyping as previously described (Li, Y. et al., Mol. Med. 18, 1312-1319. And Maminishkis, A. et al., Invst. Ophthalmol. Vis. Sci. 47, 3612-3624). All iPSC lines were passaged every 3-6 days while being maintained in mTeSR-1 medium (STEM CELL Technologies). The iPSC lines were then differentiated into RPE (iRPE) cells. To do this, they were cultured in 6-well culture plates precoated with 1:50 diluted matrigel (CORNING). For the first 2 weeks, the differentiation medium consisted of DMEM (Thermo Fisher Scientific), 15% serum replacement (Thermo Fisher Scientific), 2 mM glutamine (Thermo Fisher Scientific), 50 U/mL penicillin/streptomycin (Thermo Fisher Scientific), 1% non-essential amino acids (Thermo Fisher Scientific), and 10 mM nicotinamide (Sigma-Aldrich). For the following 2 weeks, the culture medium was supplemented with 100 ng/mL human Activin-A (PeproTech). Starting from week 5, the Activin-A was removed. The pigmented flat clusters formed in the plate were manually transferred to another matrigel-coated dish for further expansion.

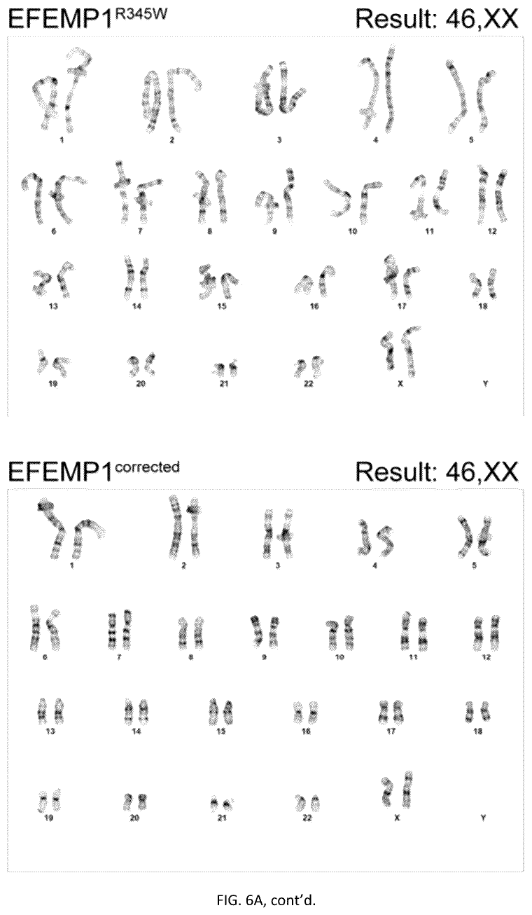

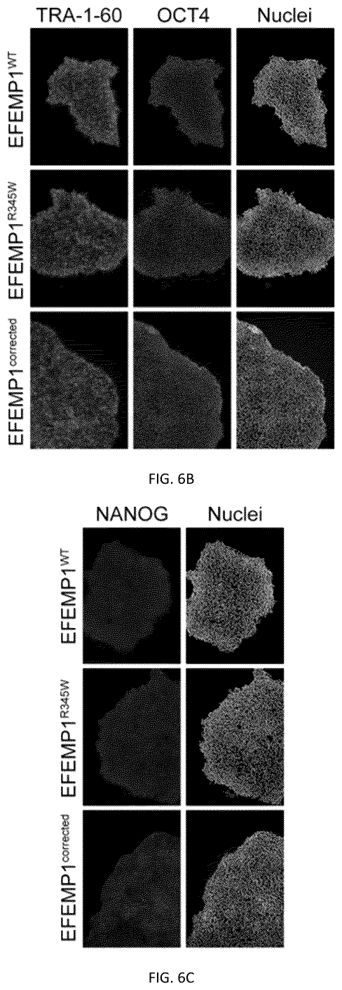

CRISPR-meditated gene correction for isogenic line To correct the p.Arg345Trp mutation in iPSCs from individuals with DHRD, cells were transfected by nucleofection using the P3 Primary Cell 4D-Nucleofector X Kit (Lonza) and the program DS150, according to the manufacturer's instructions. Briefly, a 20 μL electroporation solution was prepared for each reaction, including 15.2 μL of the P3 nucleofector solution, 3.6 μL of the supplement, and 1.2 μL of ribonucleoprotein mixture, which consists of 1 μg Cas9 protein, 300 ng gRNA, and 200 pmol single-stranded oligo donor (ssODN). iPSCs with 60%-70% confluency were used for electroporation. Accutase (Stem Cell Technology) was used to dissociate iPSCs from the plate. Approximately 2×10 5 cells were pelleted and mixed with the 20 μL electroporation solution before being transferred into the cuvette. Nucleofection was conducted on an Amaxa Nucleofector 4D. After nucleofection the cuvette, the cells were immediately transferred to one 10 cm matrigel-coated Petri dish in mTeSR1 medium with 10 μM ROCK inhibitor (Selleck Chemical). Two days later, the iPSC culture was subcultured again and split into several 10 cm Petri dishes at a density of 200 cells/dish. After 1 week of culture, iPSC colonies of appropriate size were manually picked and transferred into matrigel-coated 96-well plate for colonial expansion. The cells in each well were sampled and extracted for genomic DNA. ScrFI (New England Biolabs) was used to carry out restriction fragment length polymorphism (RFLP) assay for the screening of gene-corrected clones. The quality of iPSC lines after gene correction was confirmed by karyotyping and immunostaining (FIG. 6A-6C). Off-target site prediction was performed using Benchling webtool, and potential off-target loci in the gene-corrected clones were amplified by PCR and then analyzed by Sanger sequencing.

Retinal organoid differentiation Human iPSCs lines were maintained on Matrigel (BD)-coated plates in mTeSR medium (STEMCELL Technologies) and passaged with ReleSR (STEMCELL Technologies). Retinal organoid differentiation was carried out using the agarose microwell array seeding and scraping (AMASS) method. In brief, iPSCs at 90% confluence were detached with ReleSR (STEMCELL Technologies). After cell counting, cells were seeded at 2,000 cells per microwell (each microwell array mold contains 81 microwells) and incubated with (±)blebbistatin in mTeSR medium overnight and subsequently transitioned from mTeSR to Neural Induction Medium 1 (NIM)-1 over the next 3 days to form embryoid bodies (EBs). On differentiation day (DD) 7 EBs were transferred to Matrigel-coated wells till DD28, with a transition from NIM-1 to NIM-2 medium at DD16. Using the checkerboard-scrapping method, neuroepithelia were lifted. Once lifted, retinal organoids were maintained with NIM-2 until DD41 in poly-HEMA (Sigma)-coated wells. Retinal lamination medium 1 (RLM-1) is used from DD42 to DD69, RLM-2 from DD70 to DD97, and RLM-3 from DD98 for long-term culture. NIM1 (50 mL): 48.95 mL DMEM/F12, 10 μL 10 mg/mL heparin (final concentration, 2 μg/mL), 0.5 mL Media-Non Essential Amino Acids (100×, MEM NEAA), 0.5 mL N2 supplement (100×). NIM2 (50 mL): 48 mL DMEM/F12 (3:1), 0.5 mL MEM NEAA, 1 mL B27 Supplement (50×, minus vitamin A), 0.5 mL penicillin-streptomycin (P/S, 10,000 U/mL). RLM1 (50 mL): 42.9 mL DMEM/F12 (3:1), 0.1 mL taurine (100 μM final concentration), 5 mL FBS, 1 mL B27, 0.5 mL MEM NEAA, 0.5 mL P/S. 15. RLM2: RLM1 supplemented with 0.1 μL per mL of 10 mM retinoic acid. RLM3: RLM1 without B27, replaced with N₂ supplement and retinoic acid reduced to 0.05 μL per mL.

Comparative proteomic profiling To prepare samples for mass spectrometer analysis, each cell pellet was homogenized with 1% NP-40 lysis buffer (Thermo Fisher Scientific) with protease & phosphatase cocktails (Thermo Fisher Scientific). Enhanced BCA Protein Quantification assay (Thermo Fisher Scientific) was used to determine the total amount of protein in each sample. Proteins were further purified by mini S-trap columns (Protifi) and digested on column by trypsin. The Thermo Quantitative Fluorometric Peptide Assay was used to quantify peptide concentrations prior to TMT labeling. 40 μg peptides were labeled with TMT 6plex isobaric reagent and mixed for high pH reverse phase peptide fractionation. Thermo Orbitrap Fusion Tribrid Mass Spectrometer was used for MS/MS analysis (MS3 data acquisition method). Two iRPE lines from each group were analyzed with three biological replicates. Proteome Discoverer software (v.2.1) was used to search the acquired MS/MS data against human protein database downloaded from the UniProt website and to generate TMT ratios. Positive identification was set at 5% peptide FDR, and at least 1 unique peptide needed to be identified per protein. Duplicated protein identifications from database were removed. A total of 366 human proteins were quantified and included in the final data. TMT ratios (each tag/common reference) were calculated by PD 2.1 and normalized by total peptide amount. Qlucore Omics Explorer and Prism 6 Software were used to perform correlation and statistical analysis. KNN imputation was used for missing values.

Differentially expressed proteins (DEPs) between groups were identified by 2 fold-change and p value <0.001 using Wilcoxon signed-rank test. The DEPs were identified and visualized by volcano plots and Venn diagrams. The identified DEPs between the EFEMP1corrected and EFEMP1R345W were analyzed by gene ontology analysis via ShinyGO v.0.61, and the five most enriched groups in biological process were used to create the circus plot.

Immunofluorescence Anti-BEST1 (NB300-164, Novus Biological), anti-RPE65 (NB100-355, Novus Biological), and anti-ZO-1 (40-2300, Invitrogen) antibodies were used to detect mature RPE markers and verify differentiation by immunostaining. To detect UPR markers, anti-HSPA5 (GRP78/BIP) (Sigma) and anti-DDIT3 (CHOP) (Cell Signaling) antibodies were used. The iRPE culture was washed by PBS twice and fixed by 4% paraformaldehyde for 30 min. 5% bovine serum albumin (BSA, Thermo Fisher Scientific) was used for blocking for 2 h at room temperature. The cells were then incubated with the primary antibody diluted (1:500) with 2% BSA in PBS overnight. Alexa Fluor 488- or Alexa Fluor 555-conjugated secondary antibodies (1:500, Thermo Fisher Scientific) were used for the detection of primary antibody. Hochest staining was done at the end for counter staining.

For the detection of CES1 in human retina, human retinal paraffin sections (Biomax) were de-paraffinized with xylene for 3 min. The sections were further hydrated with 100%, 90%, 70%, and 50% alcohol for 3 min each. The sections were then incubated in running water for 10 min. To retrieve the antigen, the slides were incubated in Antigen Unmasking Solution (Thermo Fisher Scientific) for 20 min at 95° C. The slides were washed with running water for 10 min before the staining procedure.

Anti-CES1 (AF4920, R&D) and anti-EGFR (AB32077, Abcam) antibodies were diluted (1:200) with 5% BSA in PBS before being added to the slide for overnight incubation at 4° C. After 3 washes, the sections were stained with Alexa Fluor 555-conjugated secondary antibody (1:500, Thermo Fisher Scientific) for 1 h. Hochest staining was done at the end of antibody staining.

Nile red staining To stain the intracellular neutral lipid, the iRPE cell cultures were treated with 2.011 g/mL Nile red (Sigma Aldrich) for 30 min at 37° C. The cells were washed with PBS three times before microscopy observation.

Since mature iRPE culture is usually 100% confluent, the Nile red-positively stained lipid droplets were quantified using ImageJ in a fixed area of 90,000 square micrometers for the number and size of Nile red-positive signals. Fluorescent particles smaller than 0.0001 square micrometers was excluded as noise.

Cholesterol efflux assay Cholesterol Efflux Assay Kit (Abcam) was used to determine the cholesterol efflux rate in iRPE according to the manufacturer's protocol. In brief, fluorescence-labeled cholesterol was added to iRPE culture and incubated overnight. On the next day, the cells were washed twice with PBS, and the efflux of cholesterol was induced by 2% human serum. At designated time points, the culture supernatant and cell lysate were collected and measured by fluorescence at Ex/Em: 482/515 nm. The percentage of cholesterol efflux was calculated by fluorescence intensity of media fluorescence/(fluorescence intensity of cell lysate+media)×100.

Lentiviral transduction For the knockdown of CES1 (MIM: 114835) in iRPE cells, the iRPE cells from healthy donor were cultured in 6-well plate. The lentiviral particles carrying CES1 shRNA (Locus ID 1066, Origene) were transduced into the iRPE cells at a MOI of 10 overnight. To establish fair comparison, the control iRPE cells were transduced with viral particles expressing scramble shRNA. The medium was refreshed the following day, and the cell were incubated for 7 days before analysis. To achieve overexpression of CES1 in iRPE cells, lentiviral particles (Origene) expressing CMV promoter-driven CES1 (GenBank: NM_001025194) were transduced following the same procedure.

To achieve overexpression of EFEMP1^{WT} and EFEMP1^{R345W}, the lentiviral particles carrying EFEMP1^{WT} (GenBank: NM_001039348) and EFEMP1^{R345W} cDNA (Origene) were transduced into the iRPE cells at a MOI of 10 overnight. The medium was refreshed the next day, and the cell were incubated for 90 days before analysis.

Immunoblot To verify the differentiation of iRPE, CRALBP (Ab15051, Abcam) and RPE65 (401.8B11.3D9, Novas Biologicals) antibodies were used to detect mature RPE markers including CRALBP and RPE65 in iRPE cell lysates via western blot. To determine EFEMP1 expression in iRPE, both cell lysates and culture supernatant were assayed using anti-EFEMP1 antibody (MA5-25740, Thermo Fisher Scientific). To detect CES1 expression in iRPE, anti-CES1 antibody (AF4920, R&D) was used. Anti-alpha tubulin (T5168, Sigma Aldrich), anti-beta actin (A5316, Sigma), or anti-GAPDH (5174, Cell Signaling) antibodies were used to detect house-keeping proteins. The cell lysates were collected using RIPA buffer (Thermo Fisher Scientific).

To understand the upstream regulators of CES1 expression, the iRPE cells from healthy donor were treated with Gefitnib (511M, Sigma Aldrich), ML385 (1011M, Sigma Aldrich), LY294002 (10 μM, Sigma Aldrich), GW4064 (5 μM, Sigma Aldrich), or GW3965 (3 μM, Sigma Aldrich) for 72 h. Anti-CES1 (AF4920, R&D) and anti-alpha tubulin (T5168, Sigma Aldrich) antibodies were used to detect CES1 and tubulin expression, respectively. The signal was detected using Immobilon Western Chemiluminescent HRP Substrate (Millipore). The blot was quantified using ImageJ for signal intensity.

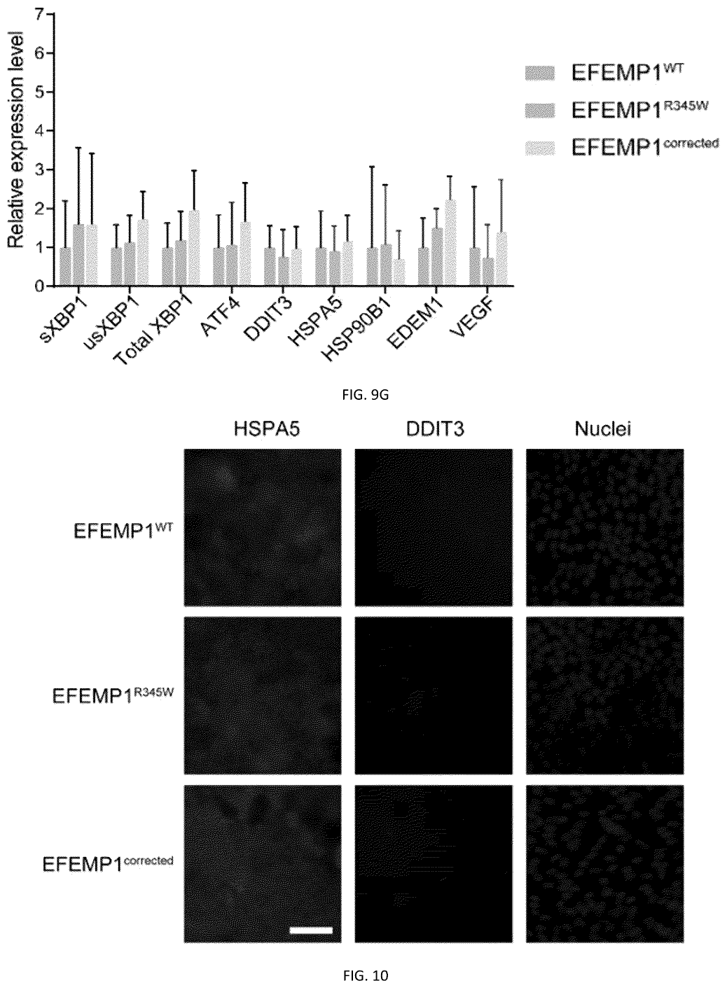

Phospho-array analysis To understand the immediate response triggered by EFEMP1^{WT} or EFEMP1^{R345W}, the iRPE cells were treated with the lysates collected from HEK293 cells transfected with either EFEMP1^{WT} cDNA, EFEMP1^{R345W} cDNA, or empty pcDNA3.1 vector by Lipofectamine 2000 (Invitrogen). Each well in a 6-well plate received 5 μg of respective plasmid. The expression of EFEMP1 protein in the lysates was confirmed by western blot (FIG. 10). The HEK293 lysate from one well was transferred into one iRPE cell culture well in a 12-well plate. After 12 h of incubation, the HEK293 lysates were washed away with PBS. The iRPE cell culture was extracted by Antibody Array Assay kit (Full Moon Biosystems). The extracts were further analyzed with EGF Pathway Phospho Antibody Array (Full Moon Biosystems) according to manufacturer's instruction.

Real-time PCR To extract mRNA from iRPE cells, the iRPE cells were harvested at the indicated time and lysed with TRIZOL reagent (Invitrogen). Total RNA was isolated according to the manufacturer's instructions. DNase I (Invitrogen) treatment was then performed to prevent genomic DNA contamination. The reverse transcription reaction was conducted by Superscript III Reverse Transcription kit, and the oligo-dT (Invitrogen) was used to generate the cDNA. Real-time PCR method was performed using SsoAdvanced Universal SYBR Green Supermix (Bio-Rad) with CFX Connect Real-Time PCR Detection System (Bio-Rad) to quantify gene expression levels. The gene expression was normalized by ACTB (MIM: 102630).

To verify that SP1 transactivates CES1, the EFEMP1^{WT} iRPE was treated with or without mithracin with a final concentration at 200 nM for 24 h before RNA extraction.

Electrophoretic mobility shift assay (EMSA) EMSA was performed to determine whether SP1 directly binds to the CES1 promoter. The EMSA was carried out using the EMSA kit (Signosis), according to manufacturer's instruction. Briefly, the iRPE nuclear extracts were collected using the Nuclear Extraction Kit (Signosis). Biotinylated DNA binding consensus sequence was also purchased from Signosis as the hot probe. 2 μg of the nuclear extract was mixed with 0.5 μL of hot probe and 1 μL of poly(I:C) for 30 min. For the competition binding experiments, 5 μL of cold probe (unlabeled oligonucleotide) were added to reaction mixtures. Samples were then loaded onto a 6.5% non-denaturing polyacrylamide gel and separated at 100 V, and the proteins were transferred to a membrane at 60 V for 1 h at 4° C. The membrane was then fixed with UV Cross-Linker (Stratagene). Streptavidin-HRP Conjugate provided by the EMSA kit to detect the signal.

Transcription factor activity assay The nuclear extracts of EFEMP1^{WT} or EFEMP1^{R345W}-treated iRPE cells were extracted using the Nuclear Extraction Kit (Signosis). TF Activation Profiling Plate Array I kit (Signosis) was used to determine the activity of transcription factors in the iRPE cells as per the manufacturer's instructions. The signal detected in the EFEMP1^{R345W}-treated iRPE cells was divided by the signal of the EFEMP1^{WT} group to reflect the change in transcription factor activity induced by the R345W mutation.

Enzyme-linked immunosorbent assay IL-1, IL-6, IL-18, TNF-alpha, TGF-beta, and IFN-alpha levels in culture supernatants were measured using IL-1 (eBioscience), IL-6 (Thermo Fisher Scientific), IL-18 kit (eBioscience), TNF-alpha (Thermo Fisher Scientific), TGF-beta (Thermo Fisher Scientific), and IFN-alpha (Origene) ELISA kits following the manufacturer's instructions. The quantification was normalized to the volume of supernatant.

Chromatin immunoprecipitation (ChIP)-qPCR assay The EFEMP1^{WT} iRPE cells were washed with PBS buffer and incubated with 1% formaldehyde at room temperature for 15 min, followed by the addition of 1.25M glycine to a final concentration of 125 mM for another 5 min. The cells were further washed by PBS buffer and lysed using ChIP assay high sensitivity kit (Abeam) following manufacturer's protocol. The SP1 antibody (Millipore, 07-645) was used for immunoprecipitation.

Statistics All statistical analysis was performed in Microsoft Excel or GraphPad Prism 8. Two-group comparisons were performed using two-tailed unpaired Student's t test. Statistical analysis for multiple group comparisons was performed using one-way ANOVA. Significance was determined at p<0.05.

### Example 1

### Gene Correction of EFEMP1 Affects EFEMP1 Secretion but not RPE Differentiation

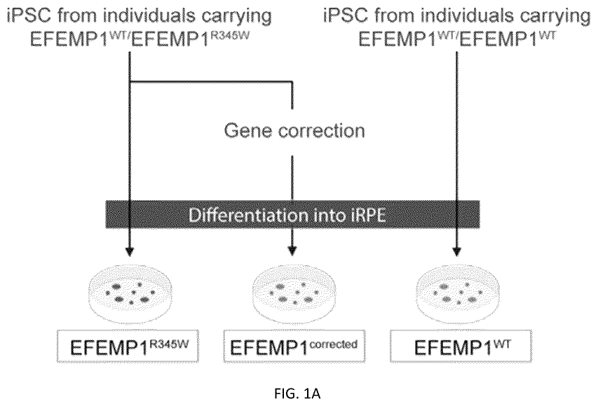

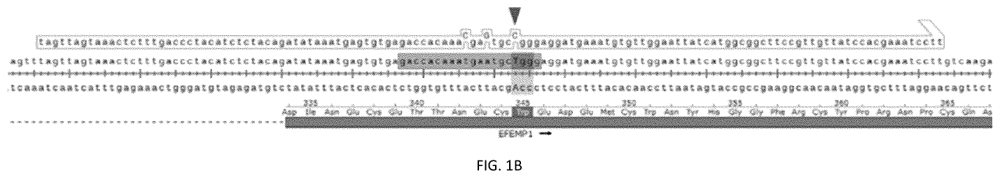

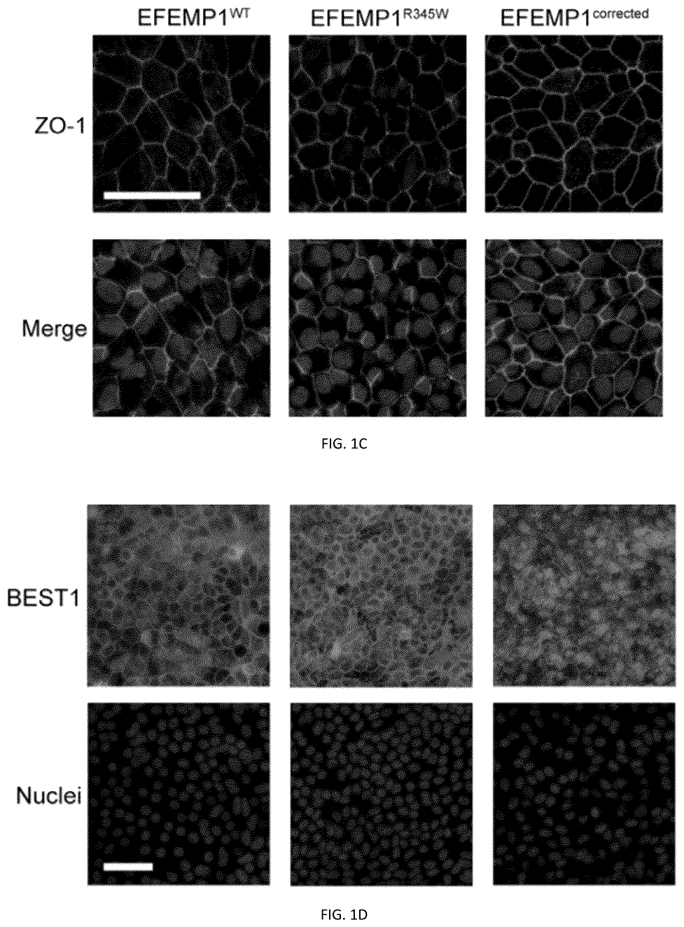

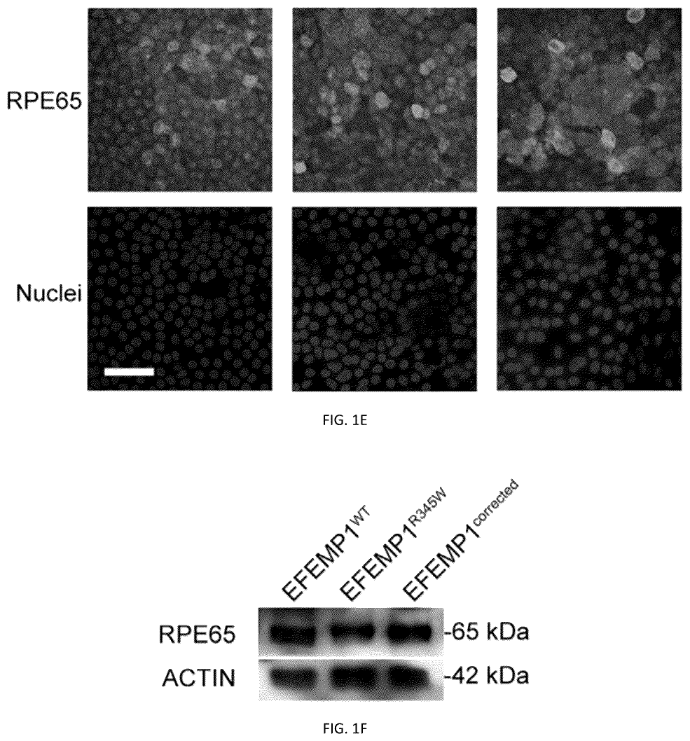

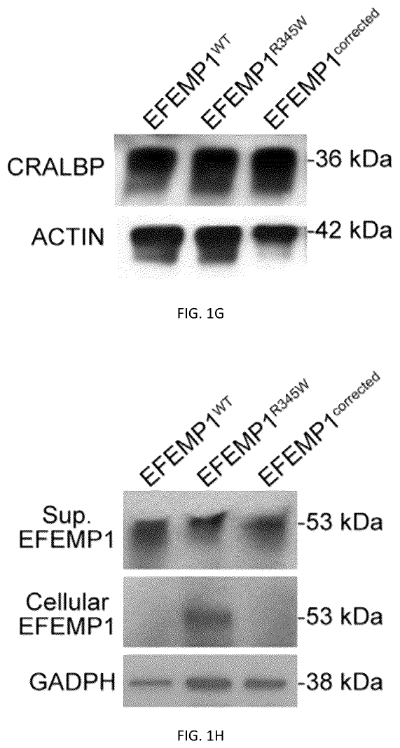

To model DHRD disease, iRPE strains were generated from three DHRD-affected individuals and healthy donor iPSCs (FIG. 1A). To minimize noise from individual difference, the CRISPR-Cas9 gene editing strategy was used to correct the pathogenic mutation (c.1033C>T mutation in EFEMP1) in the DHRD iPSCs to serve as isogenic control. The SpCas9 protein, gRNA, and donor template were delivered together into DHRD iPSCs via nucleofection to create three lines of gene-corrected iPSCs (FIG. 1B). In total, three different kinds of cell lines were created: EFEMP1^{WT} (EFEMP1^{WT}/EFEMP1^{WT}) EFEMP1^{R345W} (EFEMP1^{WT}/EFEMP1^{R345W}), and EFEMP1^{corrected} (EFEMP1^{WT}/EFEMP1^{corrected}). The EFEMP1^{WT} iRPE cells were created from iPSCs derived from healthy donor; the EFEMP1^{R345W} iRPE cells were generated using iPSCs from DHRD-affected individuals, harboring a heterozygous p.Arg345Trp mutation; while EFEMP1^{corrected} iRPE cells carry gene-corrected EFEMP1 with silent mutations. To confirm the genotypes, the wildtype, p.Arg345Trp mutant, and gene-corrected alleles were cloned into TOPO-TA vector and confirmed by sanger sequencing. RT-PCR was also used to confirm that the silent mutations do not affect normal mRNA splicing (data not shown). The quality of these iPSCs were confirmed by karyotyping and immunostaining for stem cell markers (FIGS. 6A-6C). These iPSCs were then differentiated into iRPE cells.

After differentiation, the iRPE cells from all three lines exhibited a hexagonal conformation and expressed ZO-1 in the tight junctions (FIG. 1C). The p.Arg345Trp mutation and gene correction did not alter the expression pattern of RPE markers such as BEST1 (FIG. 1D), RPE65 (FIGS. 1E and 1F), and CRALBP (FIG. 1G). In the iRPE culture, EFEMP1^{WT} protein was nearly undetectable in cell lysate but preserved in the culture supernatant (FIG. 1H). In contrast, there was an intracellular accumulation of EFEMP1^{R345W} mutant in iRPE. This disease phenotype was consistent with previous observation in DHRD-affected individual RPE staining and thus demonstrates that iRPE from DHRD-affected individuals are viable platforms for disease modeling. Notably, reduction of EFEMP1 secretion was remedied by the gene correction as evidenced by the disappearance of intracellular EFEMP1 in EFEMP1^{corrected} iRPE cells. EFEMP1 can potentially generate different isoforms with the expected size ranging from 53 kDa to 5 kDa. However, only one species of 53 kDa was detected.

### Example 2

### Differentially Expressed Genes in iRPE Cells Derived from DHRD-Affected Individuals

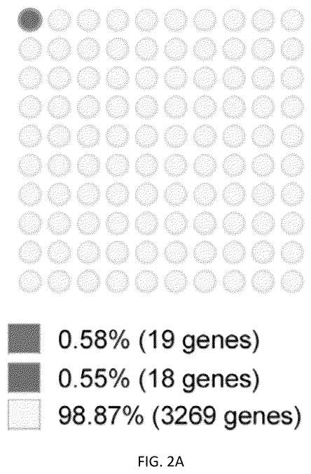

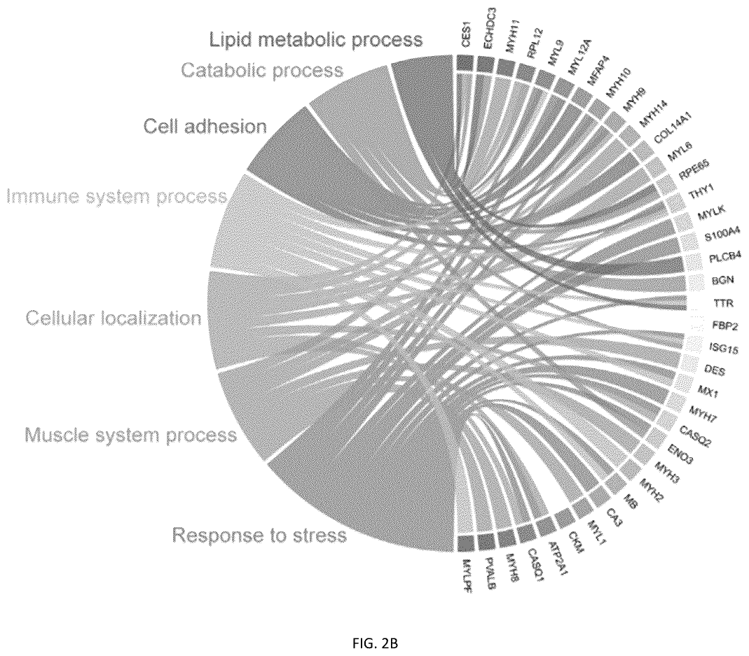

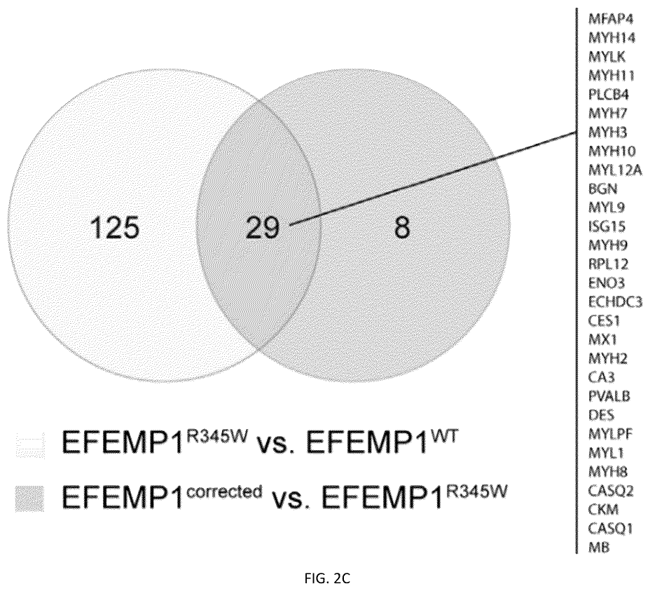

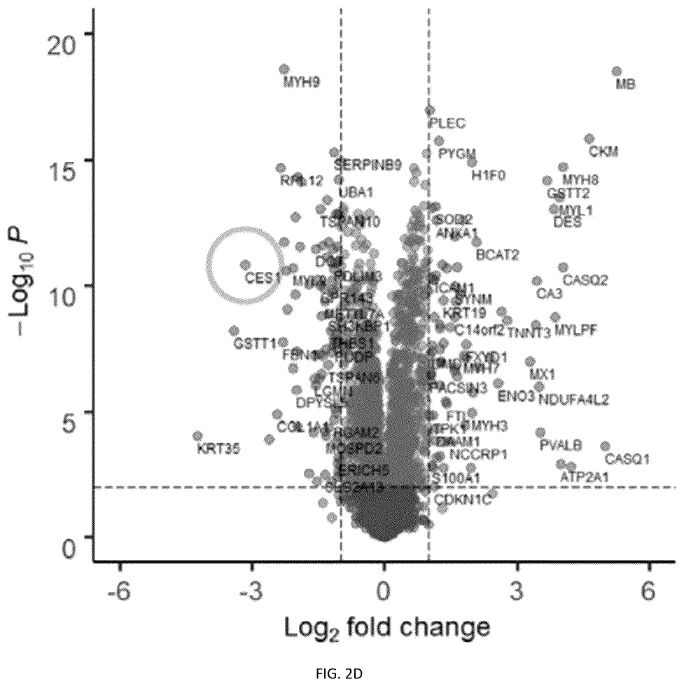

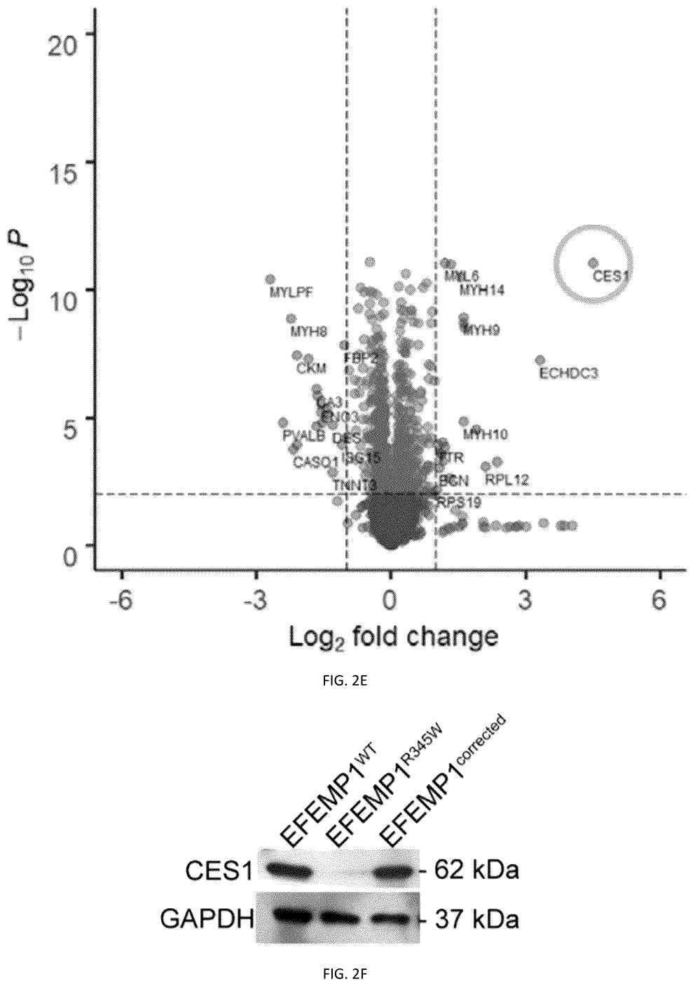

To characterize molecular changes induced by the EFEMP1^{R345W} mutation at a global level, the proteomic profiles of the EFEMP1^{WT}, EFEMP1^{R345W}, and EFEMP1^{corrected} iRPE cells were compared. All groups were analyzed in hexicate. A total of 3,269 proteins were analyzed. When EFEMP1^{WT} was compared to EFEMP1^{R345W}, 154 proteins (˜5%) were differently expressed (DEPs, fold change >2 and p value <0.001). However, when we compared EFEMP1^{R345W} iRPE clones to their isogenic controls, the number of DEP was reduced to 37 proteins (˜1%) (FIG. 2A), suggesting the validity of the isogenic controls. Of these 37 DEPs, 19 (0.58%) were upregulated and 18 (0.55%) were downregulated after gene correction.

To better understand the biological functions impacted by EFEMP1^{R345W}, the DEPs were analyzed by gene ontology (GO) analysis to identify commonly altered functions between the EFEMP1^{R345W} and EFEMP1^{corrected} cells. The most significantly enriched biological process terms among all these DEPs were response to stress, muscle system process, cellular localization, immune system process, cell adhesion, catabolic process, and lipid metabolic process (FIG. 2B). Interestingly, correction of the EFEMP1^{R345W} mutation resulted in mostly downregulation of proteins involved in immune system processes but upregulation of those participating in lipid metabolism, implicating lipid metabolism and immune system response in DHRD phenotype development. Among the 37 DEPs, 29 were shared between the EFEMP1^{WT} versus EFEMP1^{R345W} and EFEMP1^{corrected} versus EFEMP1^{R345W} comparisons (FIG. 2C).

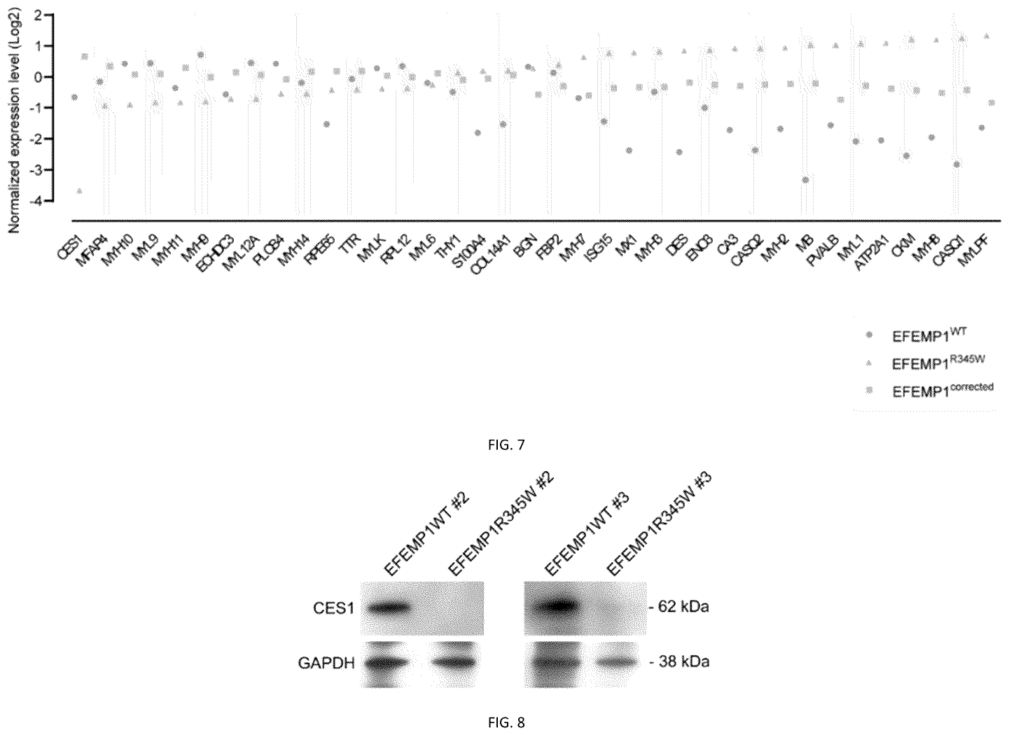

Volcano plot analysis (FIGS. 2D and 2E) was used to explore the differential protein expression signatures based on correlation between different groups. CES1 was remarkably reduced by 7- to 9-folds in EFEMP1^{R345W} cells when compared to EFEMP1^{WT} iRPE (FIGS. 2D and 7). Western blot was performed to confirm the expression of CES1 in these iRPE cells. CES1 protein can be detected in the cell lysates derived from EFEMP1^{WT} cells, but its expression was nearly undetectable in EFEMP1^{R345W} cells (FIG. 2F). This reduction of CES1 expression in DHRD iPRE was further confirmed in two other individuals with DHRD and two wild-type donor iRPE cells (FIG. 8). Strikingly, the expression level of CES1 was notably recovered after gene correction (FIGS. 2E and 2F) and comparable to the level found in EFEMP1^{WT} iRPE clones (FIG. 7). The downregulation of CES1 in EFEMP1^{R345W} cells and its restoration in EFEMP1^{connected} cells were also confirmed by immunocytochemistry (FIG. 2G).

### Example 3

### EFEMP1^{R345W} iRPE Cells Show No Inflammatory Cytokine Release or UPR Response

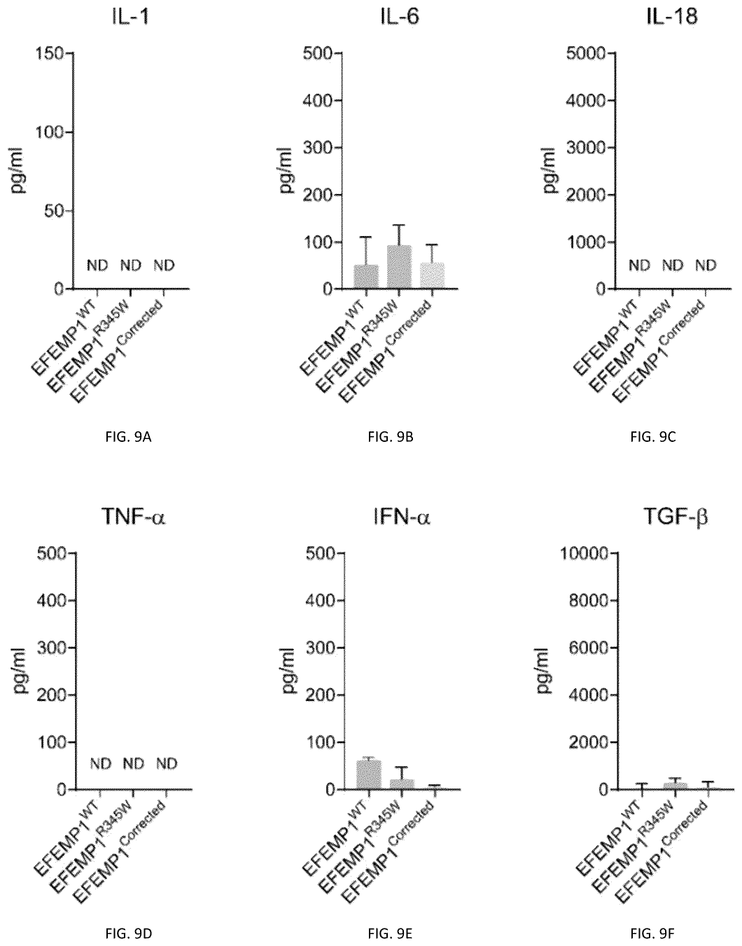

Inflammatory cytokine release has been reported in ex vivo RPE cultures of EFEMP1^{R345W} mouse. Consistently, upregulation of immune response genes such as MX1 and ISG15 was detected in our proteomic profiling (FIGS. 2B and 2C). ELISA assays were performed to further investigate whether the EFEMP1^{R345W} variant can elicit an immune response by releasing cytokines in iRPE cell culture. The medium from mature iRPE cell culture was used to detect interleukin-1 (IL-1), IL-6, IL-18, interferon alpha (IFN-α), tumor necrosis factor-alpha (TNF-α), and transforming growth factor (TGF)-beta. Interestingly, the cytokine level in all culture media was nearly undetectable, and there was no significant difference between groups (FIGS. 9A-9F).

Alternatively, it has also been suggested that the misfolded mutant EFEMP1^{R345W} protein can cause ER stress when overexpressed in a RPE cell line. To investigate this phenomenon, quantitative real-time PCR (FIG. 9G) and immunocytochemistry (FIG. 10) were carried out to detect the expression of UPR-related genes in EFEMP1^{R345W} iRPE. However, no obvious change in UPR markers was detected in EFEMP1^{R345W} iRPE clones and EFEMP1^{corrected} cells.

### Example 4

### Reduce CES1 Expression Results in Lipid Accumulation in iRPE Cells

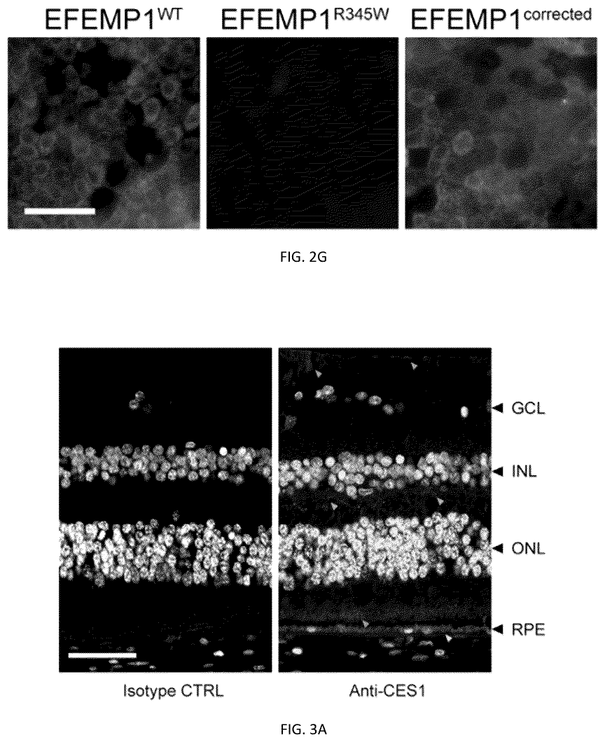

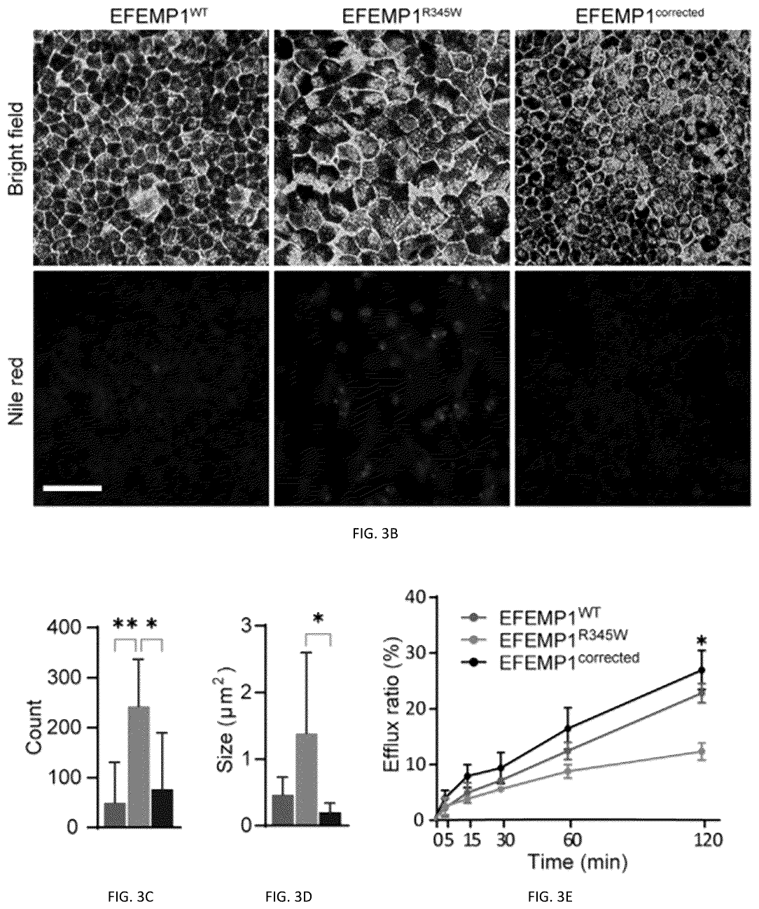

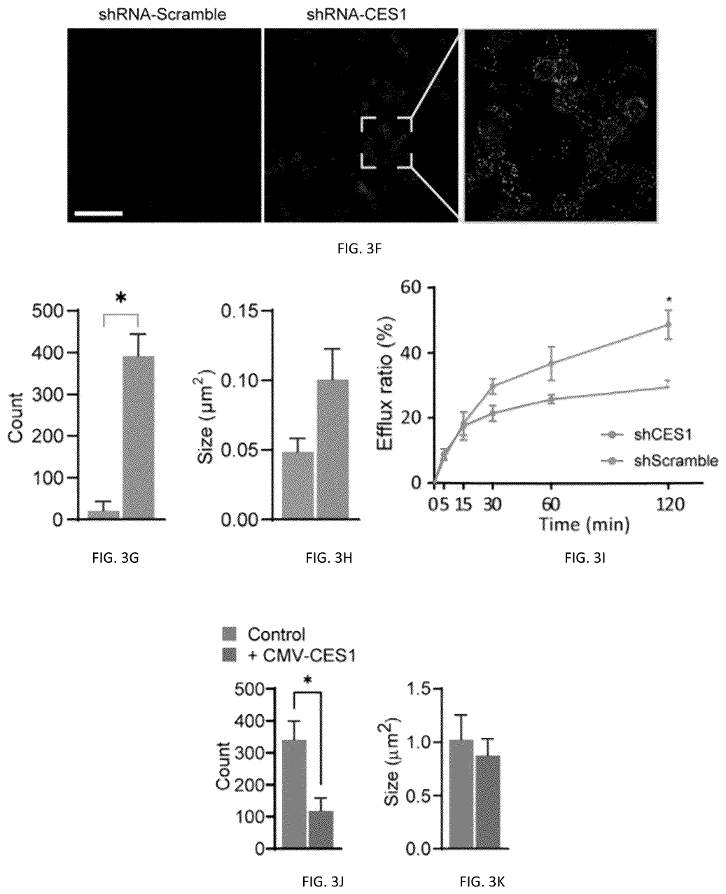

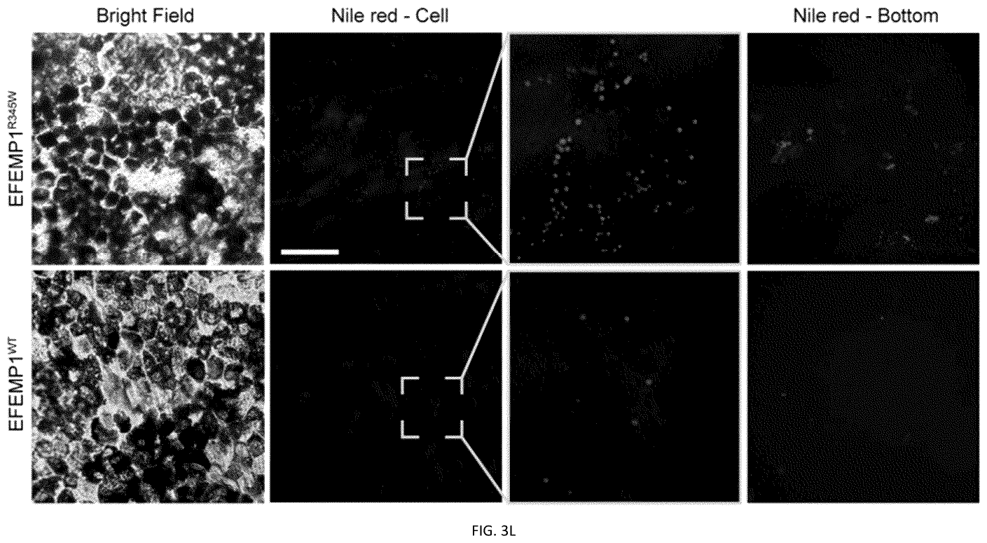

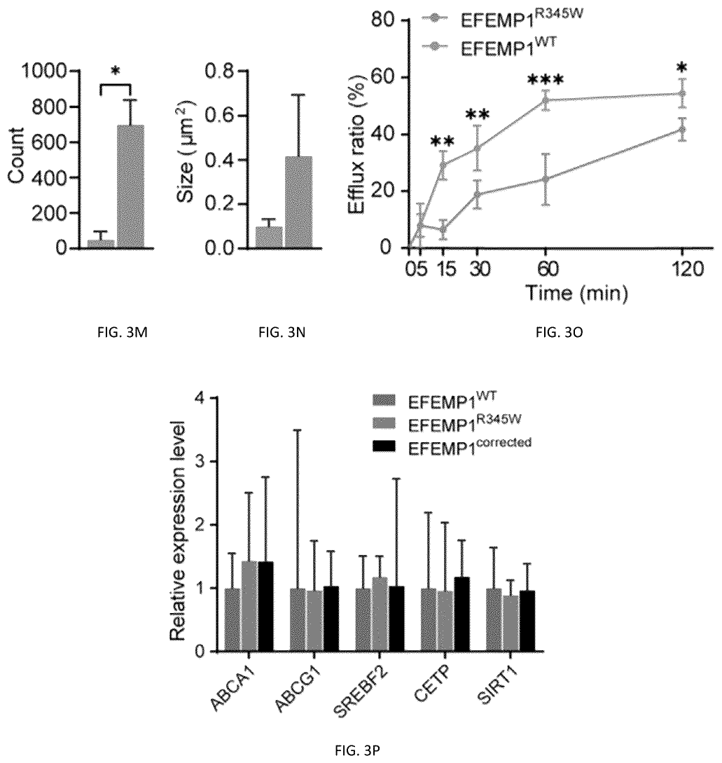

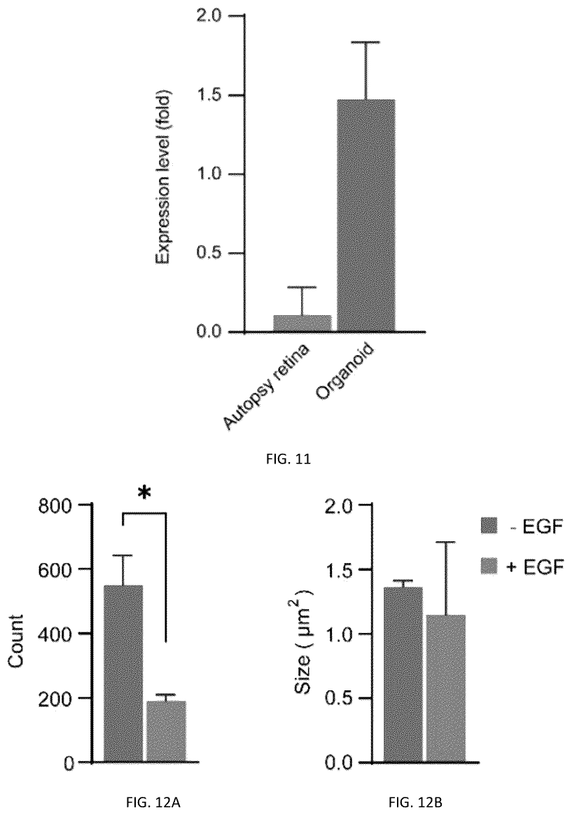

CES1 is an intracellular protein predominantly expressed in the lumen of the endoplasmic reticulum. CES1 has been reported to be expressed in hepatocytes and macrophages, but its expression in the eye remains unclear. To confirm its expression in the eye, immunostaining was performed on human eye sections. CES1 was predominantly detected in the RPE layer, but weak signal was also observed in the outer segment of photoreceptor, outer plexiform layer, and inner limiting membrane (FIG. 3A). qPCR was used to quantify the expression pattern of CES1 in retina tissue from autopsy and iPSC-derived retinal organoid. The expression of CES1 in autopsied RPE was 9.18 times higher than in neural retina (FIG. 11). However, CES1 levels in organoid retina was 1.47 times higher than in organoid RPE. These results may be attributable to the fluctuation of CES1 level during retinal development. Given that CES1 participates in converting cholesteryl ester to free cholesterol, it was hypothesized that CES1 deficiency may hamper secretion of cholesterol from RPE cells, as this hydrolytic reaction is the rate-limiting step in mobilizing stored cholesteryl ester.

To test this hypothesis, the lipid droplets in iRPE were first visualized via Nile red staining (FIG. 3B). The lipid droplets are 5 times more numerous in EFEMP1^{R345W} than in EFEMP1^{WT} iPRE (p=0.0014) (FIG. 3C) and 3 times larger in size (p=0.0727) (FIG. 3D), which indicates lipid accumulation. However, this accumulation of lipid droplets was not observed in EFEMP1^{corrected} iRPE (amount: p=0.0112; size: p=0.024). The cholesterol efflux rate of these iRPEs was determined and the EFEMP1^{R345W} iPRE exhibited <50% efflux rate when compared to either EFEMP1^{WT} or EFEMP1^{corrected} at 120 min (p=0.0197) (FIG. 3E).

To elucidate whether CES1 downregulation can result in accumulation of lipid droplet in iRPE, shRNA was used to knockdown the expression of CES1 in EFEMP1^{WT} iRPE cells. Two weeks after transduction, the lipid content in the iRPE cells was examined. In the scrambled shRNA-treated group, the signal was very weak (FIG. 3F) and comparable to that in untreated EFEMP1^{WT} iRPE cells (data not shown). However, in the CES1 knockdown group, a significant increase in the number (p=0.0115) (FIG. 3G) and slight increase in the size (p=0.0928) (FIG. 3H) of lipid droplets was observed. Moreover, shRNA-treated iRPE cells exhibited a 30%-40% decrease in cholesterol efflux compared to the scramble-treated control at 120-min time point (p=0.0318) (FIG. 3I). These results suggested that the lipid accumulation in iRPE cells were attributable to CES1 knockdown, due to a dysregulation of cholesterol efflux. To further verify the role of CES1 in lipid accumulation, CES1 was overexpressed in EFEMP1^{R345W} iRPE using lentiviral vector expressing human CES1 cDNA. After 2 weeks of transduction, though the effect in size was not obvious, the amount of lipid droplets was found to be significantly decreased 66.5% when compared to the control (p=0.0478) (FIGS. 3J and 3K). Given that CES1 is downregulated in EFEMP1^{R345W} iRPE cells, it was hypothesized that lipid accumulation can be induced by the mutant EFEMP1^{R345W} protein. To observe long-term effects, lentiviral vectors were used to transduce either a WT or p.Arg345Trp mutant copy of EFEMP1 into EFEMP1^{WT} iRPE cells. After 3 months of culture, the cells were examined by using Nile red staining. Compared to iRPE cells overexpressing WT EFEMP1, those expressing mutant EFEMP1 exhibited remarkable lipid accumulation (FIGS. 3L-3N). After removing the cells by trypsin, stained debris was observed in the dish of iRPE cells overexpressing EFEMP1^{R345W} but not EFEMP1^{WT} (FIG. 3L, rightmost column). The cholesterol efflux in these cells was tested and overexpression of EFEMP1^{R345W} reduced cholesterol efflux by 55% at 60 min (p<0.001) and by 25% at 120 min (p=0.033) (FIG. 3O).

In addition to CES1, whether there were any other cholesterol efflux-related proteins affected by the EFEMP1^{R345W} mutation that were not identified by proteomic profiling was also determined. We used real-time PCR to analyze the expression of the transporter proteins ATP-binding cassette (ABC) subfamily A member 1 (A1 [MIM: 600046]) and ABC subfamily G member 1 (G1 [MIM: 603076]), the enzymes cholesterol ester transfer protein (CETP [MIM: 118470]) and sirtuin 1 (SIRT1), and the transcription factor sterol regulatory element-binding protein 1 (SREBF2 [MIM: 601510]) in iRPE cells (FIG. 3P). No significant change in the expression of these proteins was detected in EFEMP1^{R345W} iRPE cells when compared to either EFEMP1^{WT} or EFEMP1^{corrected} clones, further supporting the causative role of CES1. Taken together, these results suggested that EFEMP1^{R345W} reduces CES1 expression and cholesterol efflux, causing lipid accumulation in iRPE cells.

### Example 5

### EFEMP1^{R345W} Regulates CES1 Expression Via EGFR-Akt Signaling

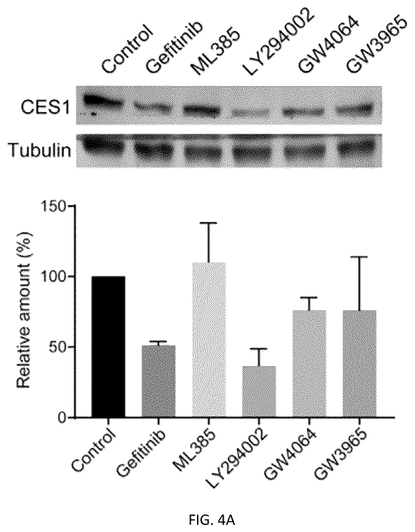

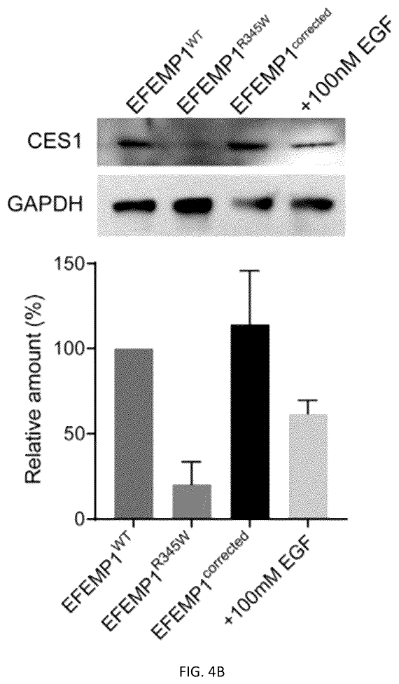

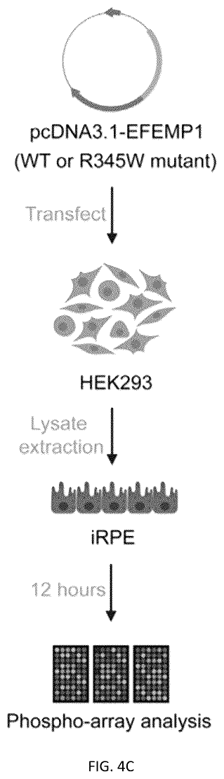

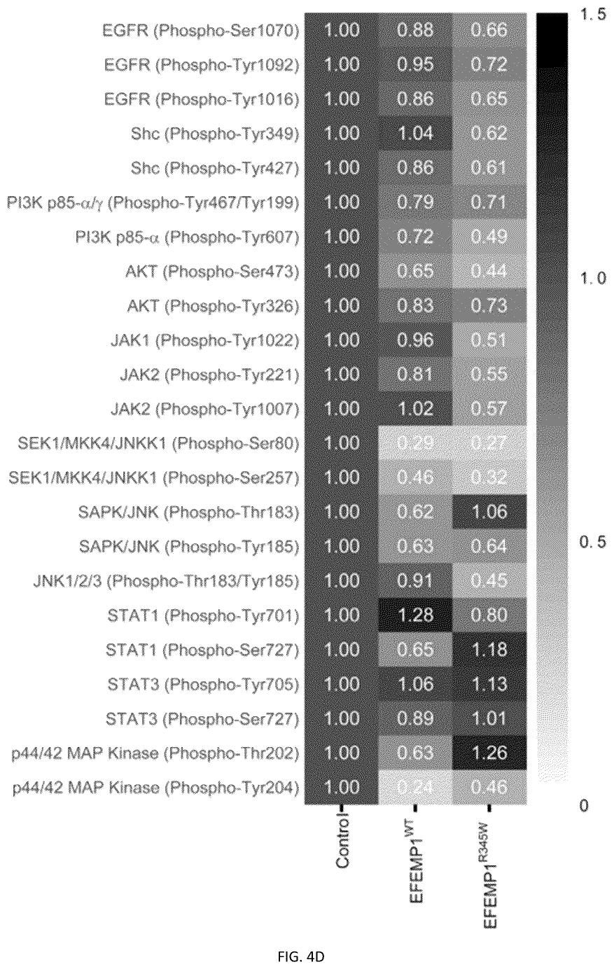

Next, we sought to understand how EFEMP1^{R345W} suppresses CES1 expression. Since EFEMP1 is an ECM protein, it was suspected that EFEMP1 may affect CES1 expression through plasma membrane receptor signaling. It has been reported that EFEMP1 can bind to EGFR directly and inhibit EGFR signaling in glioma cells. Since RPE cells express EGFR, it was hypothesized that EFEMP1 may modulate CES1 expression through EGFR signaling. EFEMP1^{WT} iRPE cells were treated with the EGFR inhibitor gefitinib. After a 72 h incubation, CES1 expression level in iRPE cells was remarkably reduced compared to untreated control, confirming that EGFR is involved in EFEMP1-induced regulation of CES1 (FIG. 4A, lanes 1 and 2).

Nuclear factor erythroid 2-related factor 2 (NRF2) and phosphatidylinositol 3-kinase (PI3K)/AKT are downstream targets of EGFR, and NRF2 has been reported to transactivate CES1 expression in the human hepatoma cell line HepG2. To determine whether either of these pathways regulate CES1 in iRPE cells, we used ML385 and LY294002 to inhibit NRF2 and PI3K/AKT, respectively. Unexpectedly, only LY294002 reduced CES1 expression (FIG. 4A, lanes 3 and 4), suggesting that the EGFR-AKT pathway is necessary for controlling CES1 levels in iRPE cells while NRF2 is not essential. Since farnesoid X receptor (FXR) and liver X receptor (LXR) were also reported to regulate CES1, EFEMP1^{WT} iRPE cells were treated with their respective agonists, GW4064 and GW3965, but no change in CES1 expression was observed (FIG. 4A, lanes 5 and 6). To further confirm EGFR signaling has positive impact on CES1 expression, EFEMP1^{R345W} iRPE were treated with 100 nM EGF. After 72 h of incubation, the CES1 level in EGF-treated iRPE became remarkably higher than that in untreated cells (FIG. 4B).

Given that the p.Arg345Trp mutation resides in the last EGF-like domain, it was hypothesized that EFEMP1^{R345W} may impact EGFR signaling. To focus on the direct effect of EFEMP1^{R345W} protein on EGFR signaling and to avoid secondary effects, EFEMP1^{WT} iRPE cells were treated with the lysates of HEK293 cells that were previously transfected with plasmids carrying either EFEMP1^{WT} or EFEMP1^{R345W} (FIGS. 4C and 10). After 12 h of incubation, the HEK293 lysate was removed. The iRPE cell lysate was extracted and analyzed using an EFGR signaling microarray. Notably, most of the downstream cascade, including PI3K/AKT1 and j anus kinase (JAK)/c-Jun N-terminal kinase (JNK), were hyper-dephosphorylated in the EFEMP1^{R345W}-treated group when compared to the EFEMP1^{WT}-treated group (FIG. 4D). These results suggest that EFEMP1^{R345W} has a hyper-inhibitory effect on EGFR signaling.

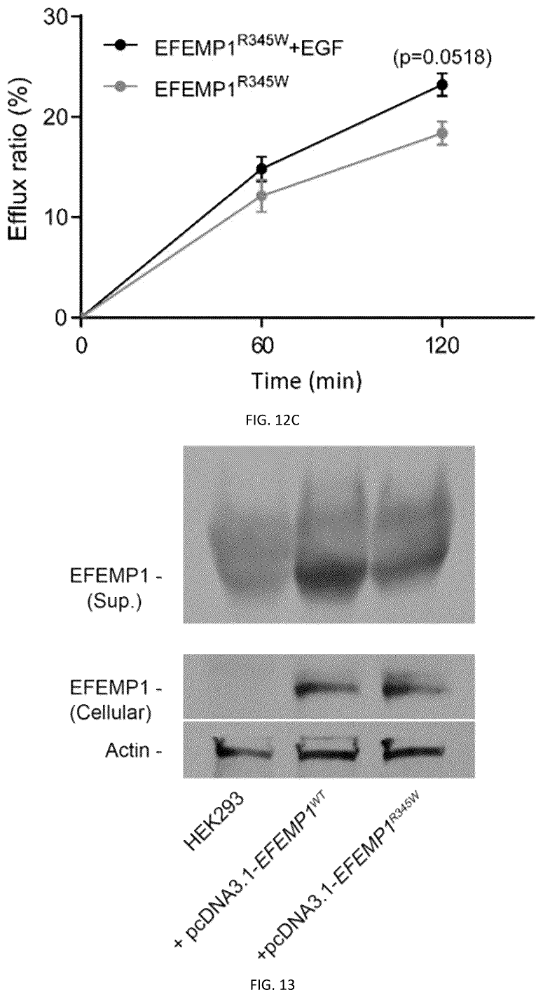

Since EGFR signaling can positively modulate CES1 expression, the treatment of 100 mM EGF on EFEMP1^{R345W} iRPE was tested. After 2 weeks of incubation, a significant decrease in the amount of lipid droplets in the cells was noted (p=0.0478) (FIG. 12A), but the effect was not obvious in the droplet size (FIG. 12B). Also observed was a 26% increase in cholesterol efflux in EGF-treated cells than in the untreated control (p=0.0518) (FIG. 12C).

### Example 6

### CES1 Transcription is Controlled by SP1

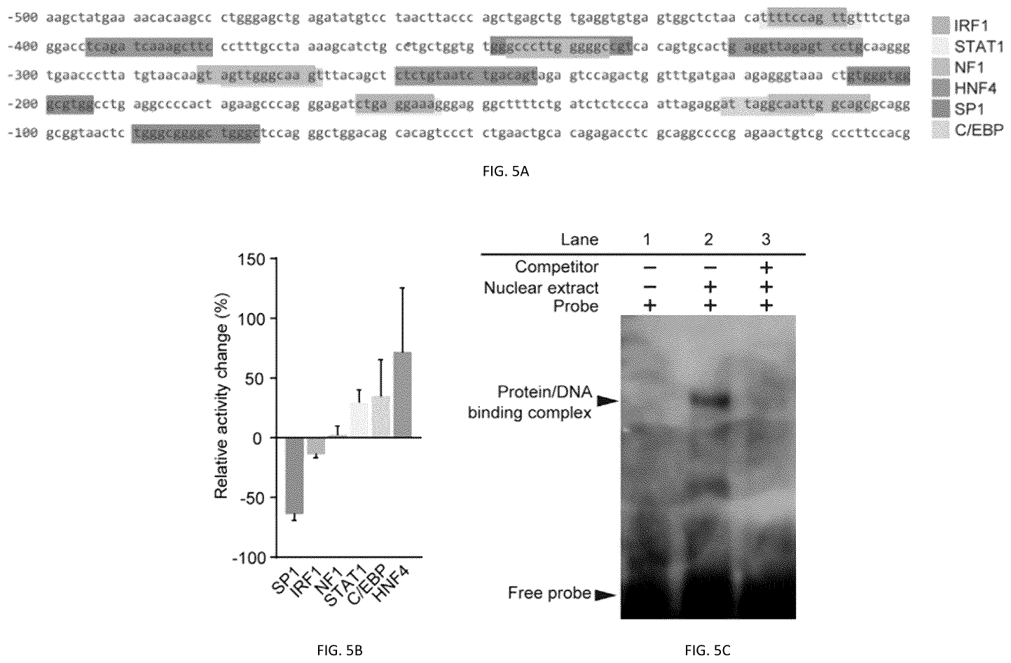

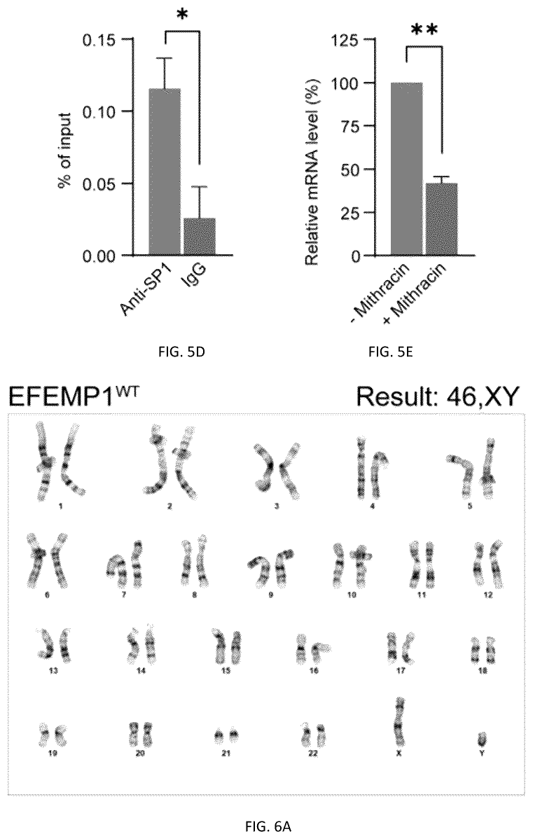

We next sought to understand how EFEMP1^{R345W}-induced reduction of EGFR signaling impacts the transcription of CES1 in iRPE cells. The proximal promoter (˜500 bp) of CES1 was observed and potential binding sites for six transcription factors were found: interferon regulator factor 1 (IRF1), signal transducer and activator of transcription 1 (STAT1), neurofibromin 1 (NF1), hepatocyte nuclear factor 4 (HNF4), SP1, and CCAAT enhancer binding protein (C/EBP) (FIG. 5A). To study the activity of these transcription factors, the nuclear extracts of EFEMP1^{WT} or EFEMP1^{R345W}-treated iRPE cells were used for analysis. SP1 activity in the EFEMP1^{R345W} group was reduced by 63.9% (FIG. 5B). In contrast, the activity of STAT1, C/EBP, and HNF4 increased by 30.0%, 35.1% and 71.9%, respectively, and there was no change in IRF1 or NF1 activity. In light of these results, it was hypothesized that EFEMP1^{R345W}-induced CES1 downregulation may be mediated by SP1.

To determine whether SP1 can bind to the CES1 promoter, EMSA was conducted using a DNA probe containing the sequence of the predicted SP1 binding site in the CES1 promoter (5′-aactgtgggtgggcgtggcctgaggcccc-3; SEQ ID NO: 1) (FIG. 5C). Incubation of this probe with nuclear extracts of EFEMP1^{WT} iRPE cells yielded a number of DNA-protein complexes on the gel (FIG. 5C, lane 1 and 2). This complex formation was eliminated by the addition of excess unlabeled probe (FIG. 5C, lane 3). Chromatin immunoprecipitation (ChIP)-qPCR was also carried out to test the binding activity of SP1 in EFEMP1^{WT} iRPE. ChIP of SP1 showed a 5 times higher enrichment than the IgG control (p=0.0387) (FIG. 5D). To further validate whether SP1 directly controls CES1 expression, EFEMP1^{WT} iRPE cells were treated with the SP1-specific inhibitor mithracin. After 24 h of incubation, expression of CES1 was reduced by 58.0% (p=0.0019) (FIG. 5E). Altogether, these results demonstrate that SP1 binds to CES1 promoter and contributes to its transactivation.

Drusen are characterized as extracellular deposits of debris that accumulate between the basal lamina of the RPE layer and the inner layer of the Bruch's membrane. On color fundus examinations, drusen manifest as small yellow-white deposits in the macular area and periphery of the retina. Drusen consist of a combination of lipids, polysaccharides, glycosaminoglycans, and proteins. Lipids, specifically, phosphatidylcholine and both esterified and unesterified cholesterol, are thought to comprise the bulk of drusen. Consistently, extracellular proteins responsible for cholesterol mobilization including apolipoprotein B and E have also been closely associated with drusen. Nonetheless, the specific mechanism underlying drusen formation remains unclear.

The presence of drusen is a clinical hallmark of various forms of MD including DHRD and AMD. The phenotypic similarity between these two diseases suggests that they share the same pathology. However, unlike AMD's heterogenous etiology, DHRD is monogenic and thus offers a more approachable experimental model for drusen pathogenesis. Prior studies investigating DHRD have suggested two possible mechanisms for how mutant EFEMP1 contributes to drusen formation: the UPR and complement system activation. Roybal et al. (Invest. Ophthalmol. Vis. Sci. 2005; 46:3973-3979) found that overexpression of EFEMP1^{R345W} in ARPE-19 elicited UPR activation and increased VEGF expression. In contrast, using EFEMP1^{R345W/R345W} mice as an in vivo disease model, Fu et al. (Hum. Mol. Genet. 2007; 16:2411-2422) found no UPR activation as measured by Hspa5 (Grp78) expression, but instead detected activated complement component C3 in the RPE and Bruch's membrane. More recently, Fernandez-Godino et al. (Hum. Mol. Genet. 2015; 24:5555-5569) suggested that increased production of C3a in EFEMP1^{R345W/R345W} mice stimulated the release of cytokines IL-6 and IL-1B.

Here, iRPE derived from three distinct individuals with DHRD were used as an in vitro disease model and leveraged isogenic comparisons to investigate how the EFEMP1^{R345W} variant contributes to DHRD. Conventional study designs investigating DHRD pathology have used family members as control subjects. However, family members are not genetically identical, and different single-nucleotide polymorphisms (SNPs) can impact disease expressivity. Engineering an isogenic cell line from the same parental line removes noise from the genetic background and yields an opportunity to elucidate specific molecular mechanisms and pathways underlying disease.

Given that previous studies implicated UPR and the complement pathway in DHRD, it was sought after to reproduce and validate those claims. Interestingly, no inflammatory cytokine release or UPR was detected in the DHRD iRPE cell culture. One possible explanation for this discrepancy may lie in the limited translatability of the disease models used in the previous studies as overexpression of the EFEMP1^{R345W} variant and homozygous p.Arg345Trp mutations do not mimic most cases of DHRD, which are caused by a single heterozygous mutation. Though no inflammatory cytokines or chemokines were detected in the in vitro iRPE model, immune response may still play a role in DHRD development in vivo. An EFEMP1-humanized model with p.Arg345Trp mutation in mouse or other animal models may offer additional insights and clarifications into this question.

An unbiased genetic approach was used to determine DEPs in our iRPE model.

Three iRPE cell comparisons: (1) EFEMP1^{R345W} versus EFEMP1^{WT}; (2) EFEMP1^{R345W} versus EFEMP1^{corrected}; and (3) EFEMP1^{R345W} versus EFEMP1^{WT} were performed. Notably, the number of DEPs decreased significantly in the comparisons involving isogenic controls. Only 37 proteins (1.13% of the total number of screened proteins) exhibited differential expression between EFEMP1^{R345W} and its isogenic control EFEMP1^{corrected}. The number of DEPs was further reduced to eight proteins when comparing EFEMP1^{R345W} to both EFEMP1^{corrected} and EFEMP1^{WT} iRPE cells. Overall, the isogenic control helped exclude around 80%-95% of the genetic noise. Importantly, the protein expression patterns of the 37 DEPs in EFEMP1^{corrected} iRPE cells were more similar to those of EFEMP1^{WT} iRPE cells than to those of DHRD iRPE cells, indicating the involvement of these 37 proteins in the pathogenesis of DHRD.

GO was then used to analyze the 37 DEGs and grouped these genes into enriched terms, including lipid metabolic process, catabolic process, cell adhesion, immune system process, cellular localization, muscle system process, and response to stress. Based on current literature, most of these genes do not directly participate in cholesterol metabolism. Nonetheless, gene correction induced a significant increase in certain genes including CES1, phospholipase C beta 4 (PLCB4), and RPE65, all of which participate in lipid metabolic or catabolic processes. CES1 is responsible for the hydrolysis of ester- and amide-bond-containing xenobiotics as well as long-chain fatty acid esters and thioesters. CES1 acts as a rate-limiting enzyme in reverse cholesterol transport—specifically, the conversion of cholesteryl ester to free cholesterol—in macrophages during regression of atherosclerosis. PLCB4 catalyzes the formation of inositol 1,4,5-trisphosphate and diacylglycerol from phosphatidylinositol 4,5-bisphosphate, an important reaction in intracellular transduction of extracellular signals in the retina. RPE65 converts all-trans-retinyl esters to 11-cis-retinol, the rate limiting step for the retinoid cycle.

CES1 was the focus because CES1 exhibited the largest change between DHRD and healthy iRPE cells. Gene correction increased the expression level of CES1 by ˜20-fold compared to that of diseased iRPE cells from either of the two DHRD-affected individuals. Interestingly, CES1 levels in EFEMP1^{corrected} was ˜3-fold higher than in EFEMP1^{WT} cells, indicating a non-trivial variation from endogenous CES1 expression. This could explain the variability and partial penetrance of DHRD. The role of CES1 has not been well studied in RPE. However, in macrophage/foam cells, CES1 has been reported to play a major role in reverse cholesterol transport during atherosclerosis regression. RPE cells resemble macrophages in their roles in active phagocytosis and cholesterol efflux. RPE cells are the most actively phagocytic cells in the human body. Within the retina, RPE cells are responsible for phagocytosing shed photoreceptor outer segments and preventing subsequent accumulation of excess lipid via lipid export. It is therefore possible that RPE cells and macrophages share the same pathways/mechanisms for controlling cholesterol transport.

Notably, it was discovered that downregulation of CES1 leads to abnormal lipid accumulation in iRPE cells. Strikingly, EFEMP1^{R345W} had a hyper-inhibitory effect on EGFR signaling, possibly by deactivating PI3K/AKT signaling. EGFR signaling was associated with increased expression of CES1 in iRPE cells. Taking these results together, the following model is proposed: the p.Arg345Trp mutation enhances EFEMP1's inhibitory activity on the EGFR-Akt pathway, downregulating CES1. Reduced expression of CES1 disrupts cholesterol efflux and leads to abnormal lipid accumulation in the RPE cells. This model is consistent with the current understanding of MD pathology. Cholesterol efflux genes such as apolipoprotein E (APOE) and ABCA1 have been identified as risk factors for AMD, and deficiency in cholesterol efflux is thought to accelerate AMD progression by promoting deposition of drusen and other extracellular lipids underneath the retina. Interestingly, the expression level of EFEMP1 was recently found to increase with age. Given that EFEMP1 inhibits the expression of CES1, aging may decrease cholesterol efflux in RPE cells by perturbing ECM homeostasis.

Lipid metabolism and immune responses are closely integrated through converging pathways in many tissues. Previous studies have demonstrated that the absence of infectious diseases and prolonged nutrient excess induce chronic low-level sterile metaflammation through immune pathway activation. Consistent with these observations, the GO analysis revealed that a number of downregulated proteins, including ISG15 and MX1, participates in immune response. While all of these proteins can be induced by interferons, few anti-viral cytokine secretions were detected in our iRPE cell culture. One possible explanation for this phenomenon is that the high expression of immune-related genes in DHRD iRPE may be an aftermath of the excessive accumulation of cytoplasmic lipids. Metabolic disorders involve a complex interplay of dysregulated pathways, leading to lipotoxicity, inflammation, and associated stress responses. This intricate dynamic makes it challenging to elucidate the exact mechanism of disease and identify interventional targets. One finding is that SP1 binds to the CES1 promoter. As a zinc finger transcription factor, SP1 is involved in many different cellular processes including immune response.

In this study lipid deposits were observed after the iRPE were removed from the polystyrene cell culture plate. This phenomenon was also observed in a previous study, where iRPE cultures were grown on Transwell membrane. Drusen has been previously shown to contain high levels of lipids such as cholesteryl ester and phosphatidylcholine. This finding suggests that iRPE may produce drusen-like sub-RPE deposit in a cell-autologous fashion. There was no sign of massive cell death in the EFEMP1^{R345W} iRPE cell culture by the 3-month endpoint of the experiment (data not shown), and there was no further investigation of the effect of aberrant lipid accumulation on the iRPE cells. However, lipid accumulation in RPE cells is believed to be harmful if persistent. For example, excessive lipids may perturb calcium homeostasis by increasing ER or mitochondrial stress. This intracellular lipotoxicity may eventually contribute to RPE atrophy which has been observed in DHRD- and AMD-affected individuals. If lipotoxicity kills cells, cholesteryl ester together with dead cell debris may remain precipitated around the RPE/BrM and become drusen. As such, the resultant formation of drusen may be partially attributed to the immobility of cholesteryl ester. Its clearance, in turn, must be achieved through ingestion by another cell (e.g., macrophage). This hypothesis might explain why esterified cholesterol, which usually exists intracellularly, constitutes a large part of drusen.

In this study it was shown that accumulation of intracellular cholesterol ester in DHRD-affected individual-derived RPE cells is a result of dysregulated cholesterol efflux. Cholesterol imbalance can also be exacerbated by increased uptake of lipids. Whether a low-fat diet as a supplement to gene editing-based therapy can postpone the onset of drusen or ameliorate drusen severity is an interesting direction for the development of treatments for not only DHRD but also other forms of MD such as AMD.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

### REFERENCES

1. Pennington K. L., DeAngelis M. M. Epidemiology of age-related macular degeneration (AMD):

associations with cardiovascular disease phenotypes and lipid factors. Eye Vis. (Lond.) 2016; 3:34.
2. Bryan J. A., 3rd, Campochiaro P. A. A retinal pigment epithelial cell-derived growth factor(s)

Arch. Ophthalmol. 1986; 104:422-425.
3. Karamali F., Esfahani M. N., Hajian M., Ejeian F., Satarian L., Baharvand H. Hepatocyte growth factor promotes the proliferation of human embryonic stem cell derived retinal pigment epithelial cells. J. Cell. Physiol. 2019; 234:4256-4266.
4. Sarks S., Cherepanoff S., Killingsworth M., Sarks J. Relationship of Basal laminar deposit and membranous debris to the clinical presentation of early age-related macular degeneration. Invest. Ophthalmol. Vis. Sci. 2007; 48:968-977.
5. Anderson D. H., Mullins R. F., Hageman G. S., Johnson L. V. A role for local inflammation in the formation of drusen in the aging eye. Am. J. Ophthalmol. 2002; 134:411-431.
6. Fernandez-Godino R., Pierce E. A. C3a triggers formation of sub-retinal pigment epithelium deposits via the ubiquitin proteasome pathway. Sci. Rep. 2018; 8:9679.
7. Seddon J. M., Yu Y., Miller E. C., Reynolds R., Tan P. L., Gowrisankar S., Goldstein J. I., Triebwasser M., Anderson H. E., Zerbib J. Rare variants in CFI, C3 and C9 are associated with high risk of advanced age-related macular degeneration. Nat. Genet. 2013; 45:1366-1370.
8. Bressler N. M., Bressler S. B., Fine S. L. Age-related macular degeneration. Surv. Ophthalmol. 1988; 32:375-413.
9. de Jong P. T. Age-related macular degeneration. N. Engl. J. Med. 2006; 355:1474-1485.
10. Jager R. D., Mieler W. F., Miller J. W. Age-related macular degeneration. N. Engl. J. Med. 2008; 358:2606-2617.
11. Haimovici R., Wroblewski J., Piguet B., Fitzke F. W., Holder G. E., Arden G. B., Bird A. C. Symptomatic abnormalities of dark adaptation in patients with EFEMP1 retinal dystrophy (Malattia Leventinese/Doyne honeycomb retinal dystrophy) Eye (Lond.) 2002; 16:7-15.
12. Michaelides M., Jenkins S. A., Brantley M. A., Jr., Andrews R. M., Waseem N., Luong V., Gregory-Evans K., Bhattacharya S. S., Fitzke F. W., Webster A. R. Maculopathy due to the R345W substitution in fibulin-3: distinct clinical features, disease variability, and extent of retinal dysfunction. Invest. Ophthalmol. Vis. Sci. 2006; 47:3085-3097.
13. Hulleman J. D. Malattia Leventinese/Doyne Honeycomb Retinal Dystrophy: Similarities to Age-Related Macular Degeneration and Potential Therapies. Adv. Exp. Med. Biol. 2016; 854:153-158.
14. Klein M. L., Schultz D. W., Edwards A., Matise T. C., Rust K., Berselli C. B., Trzupek K., Weleber R. G., Ott J., Wirtz M. K., Acott T. S. Age-related macular degeneration. Clinical features in a large family and linkage to chromosome 1 q. Arch. Ophthalmol. 1998; 116:1082-1088.
15. Schultz D. W., Weleber R. G., Lawrence G., Banal S., Majewski J., Acott T. S., Klein M. L. HEMICENTIN-1 (FIBULIN-6) and the 1q31 AMD locus in the context of complex disease: review and perspective. Ophthalmic Genet. 2005; 26:101-105.
16. Stone E. M., Braun T. A., Russell S. R., Kuehn M. H., Lotery A. J., Moore P. A., Eastman C. G., Casavant T. L., Sheffield V. C. Missense variations in the fibulin 5 gene and age-related macular degeneration. N. Engl. J. Med. 2004; 351:346-353.
17. Stone E. M., Lotery A. J., Munier F. L., Ekon E., Piguet B., Guymer R. H., Vandenburgh K., Cousin P., Nishimura D., Swiderski R. E. A single EFEMP1 mutation associated with both Malattia Leventinese and Doyne honeycomb retinal dystrophy. Nat. Genet. 1999; 22:199-202.
18. Thompson C. L., Klein B. E., Klein R., Xu Z., Capriotti J., Joshi T., Leontiev D., Lee K. E., Elston R. C., Iyengar S. K. Complement factor H and hemicentin-1 in age-related macular degeneration and renal phenotypes. Hum. Mol. Genet. 2007; 16:2135-2148.
19. McLaughlin P. J., Bakall B., Choi J., Liu Z., Sasaki T., Davis E. C., Marmorstein A. D., Marmorstein L. Y. Lack of fibulin-3 causes early aging and herniation, but not macular degeneration in mice. Hum. Mol. Genet. 2007; 16:3059-3070.
20. Fu L., Garland D., Yang Z., Shukla D., Rajendran A., Pearson E., Stone E. M., Zhang K., Pierce E. A. The R345W mutation in EFEMP1 is pathogenic and causes AMD-like deposits in mice. Hum. Mol. Genet. 2007; 16:2411-2422.
21. Marmorstein L. Y., McLaughlin P. J., Peachey N. S., Sasaki T., Marmorstein A. D. Formation and progression of sub-retinal pigment epithelium deposits in Efemp1 mutation knock-in mice: a model for the early pathogenic course of macular degeneration. Hum. Mol. Genet. 2007; 16:2423-2432.
22. Hulleman J. D., Kaushal S., Balch W. E., Kelly J. W. Compromised mutant EFEMP1 secretion associated with macular dystrophy remedied by proteostasis network alteration. Mol. Biol. Cell. 2011; 22:4765-4775.
23. Marmorstein L. Y., Munier F. L., Arsenijevic Y., Schorderet D. F., McLaughlin P. J., Chung D., Traboulsi E., Marmorstein A. D. Aberrant accumulation of EFEMP1 underlies drusen formation in Malattia Leventinese and age-related macular degeneration. Proc. Natl. Acad. Sci. USA. 2002; 99:13067-13072.
24. Fernandez-Godino R., Garland D. L., Pierce E. A. A local complement response by RPE causes early-stage macular degeneration. Hum. Mol. Genet. 2015; 24:5555-5569.
25. Fernandez-Godino R., Bujakowska K. M., Pierce E. A. Changes in extracellular matrix cause RPE cells to make basal deposits and activate the alternative complement pathway. Hum. Mol. Genet. 2018; 27:147-159.
26. Roybal C. N., Marmorstein L. Y., Vander Jagt D. L., Abcouwer S. F. Aberrant accumulation of fibulin-3 in the endoplasmic reticulum leads to activation of the unfolded protein response and VEGF expression. Invest. Ophthalmol. Vis. Sci. 2005; 46:3973-3979.
27. Li Y., Tsai Y. T., Hsu C. W., Erol D., Yang J., Wu W. H., Davis R. J., Egli D., Tsang S. H. Long-term safety and efficacy of human-induced pluripotent stem cell (iPS) grafts in a preclinical model of retinitis pigmentosa. Mol. Med. 2012; 18:1312-1319.
28. Maminishkis A., Chen S., Jalickee S., Banzon T., Shi G., Wang F. E., Ehalt T., Hammer J. A., Miller S. S. Confluent monolayers of cultured human fetal retinal pigment epithelium exhibit morphology and physiology of native tissue. Invest. Ophthalmol. Vis. Sci. 2006; 47:3612-3624.
29. Cowan C. S., Renner M., De Gennaro M., Gross-Scherf B., Goldblum D., Hou Y., Munz M., Rodrigues T. M., Krol J., Szikra T. Cell Types of the Human Retina and Its Organoids at Single-Cell Resolution. Cell. 2020; 182:1623-1640.e34.
30. Lian J., Nelson R., Lehner R. Carboxylesterases in lipid metabolism: from mouse to human. Protein Cell. 2018; 9:178-195.
31. Xu J., Li Y., Chen W. D., Xu Y., Yin L., Ge X., Jadhav K., Adorini L., Zhang Y. Hepatic carboxylesterase 1 is essential for both normal and farnesoid X receptor-controlled lipid homeostasis. Hepatology. 2014; 59:1761-1771.
32. Zhao B., Song J., Ghosh S. Hepatic overexpression of cholesteryl ester hydrolase enhances cholesterol elimination and in vivo reverse cholesterol transport. J. Lipid Res. 2008; 49:2212-2217.
33. Ghosh S. Macrophage cholesterol homeostasis and metabolic diseases: critical role of cholesteryl ester mobilization. Expert Rev. Cardiovasc. Ther. 2011; 9:329-340.
34. Hu Y., Gao H., Vo C., Ke C., Pan F., Yu L., Siegel E., Hess K. R., Linskey M. E., Zhou Y. H. Anti-EGFR function of EFEMP1 in glioma cells and patient prognosis. Oncoscience. 2014; 1:205-215.
35. Yan F., Hui Y. N., Li Y. J., Guo C. M., Meng H. Epidermal growth factor receptor in cultured human retinal pigment epithelial cells. Ophthalmologica. 2007; 221:244-250.
36. Maruichi T., Fukami T., Nakajima M., Yokoi T. Transcriptional regulation of human carboxylesterase 1A1 by nuclear factor-erythroid 2 related factor 2 (Nrf2) Biochem. Pharmacol. 2010; 79:288-295.
37. Mullins R. F., Hageman G. S. Human ocular drusen possess novel core domains with a distinct carbohydrate composition. J. Histochem. Cytochem. 1999; 47:1533-1540.
38. Abdelsalam A., Del Priore L., Zarbin M. A. Drusen in age-related macular degeneration: pathogenesis, natural course, and laser photocoagulation-induced regression. Surv. Ophthalmol. 1999; 44:1-29.
39. Curcio C. A., Presley J. B., Malek G., Medeiros N. E., Avery D. V., Kruth H. S. Esterified and unesterified cholesterol in drusen and basal deposits of eyes with age-related maculopathy. Exp. Eye Res. 2005; 81:731-741.
40. Curcio C. A., Presley J. B., Millican C. L., Medeiros N. E. Basal deposits and drusen in eyes with age-related maculopathy: evidence for solid lipid particles. Exp. Eye Res. 2005; 80:761-775.
41. Hageman G. S., Luthert P. J., Victor Chong N. H., Johnson L. V., Anderson D. H., Mullins R. F. An integrated hypothesis that considers drusen as biomarkers of immune-mediated processes at the RPE-Bruch's membrane interface in aging and age-related macular degeneration. Prog. Retin. Eye Res. 2001; 20:705-732.
42. Hageman G. S., Mullins R. F. Molecular composition of drusen as related to substructural phenotype. Mol. Vis. 1999; 5:28.
43. Hageman G. S., Mullins R. F., Russell S. R., Johnson L. V., Anderson D. H. Vitronectin is a constituent of ocular drusen and the vitronectin gene is expressed in human retinal pigmented epithelial cells. FASEB J. 1999; 13:477-484.
44. Jiang K., To E., Cui J. Z., Cao S., Gao J., Matsubara J. A. Drusen and Pro-inflammatory Mediators in the Post-Mortem Human Eye. J. Clin. Exp. Ophthalmol. 2012; 3:208.
45. Kamei M., Hollyfield J. G. TIMP-3 in Bruch's membrane: changes during aging and in age-related macular degeneration. Invest. Ophthalmol. Vis. Sci. 1999; 40:2367-2375.
46. Luibl V., Isas J. M., Kayed R., Glabe C. G., Langen R., Chen J. Drusen deposits associated with aging and age-related macular degeneration contain nonfibrillar amyloid oligomers. J. Clin. Invest. 2006; 116:378-385.
47. Malek G., Li C. M., Guidry C., Medeiros N. E., Curcio C. A. Apolipoprotein B in cholesterol-containing drusen and basal deposits of human eyes with age-related maculopathy. Am. J. Pathol. 2003; 162:413-425.
48. Mullins R. F., Russell S. R., Anderson D. H., Hageman G. S. Drusen associated with aging and age-related macular degeneration contain proteins common to extracellular deposits associated with atherosclerosis, elastosis, amyloidosis, and dense deposit disease. FASEB J. 2000; 14:835-846.
49. Wang L., Clark M. E., Crossman D. K., Kojima K., Messinger J. D., Mobley J. A., Curcio C. A. Abundant lipid and protein components of drusen. PLoS ONE. 2010; 5:e10329.
50. Curcio C. A., Millican C. L., Bailey T., Kruth H. S. Accumulation of cholesterol with age in human Bruch's membrane. Invest. Ophthalmol. Vis. Sci. 2001; 42:265-274.
51. Haimovici R., Gantz D. L., Rumelt S., Freddo T. F., Small D. M. The lipid composition of drusen, Bruch's membrane, and sclera by hot stage polarizing light microscopy. Invest. Ophthalmol. Vis. Sci. 2001; 42:1592-1599.
52. Pauleikhoff D., Zuels S., Sheraidah G. S., Marshall J., Wessing A., Bird A. C. Correlation between biochemical composition and fluorescein binding of deposits in Bruch's membrane. Ophthalmology. 1992; 99:1548-1553.
53. Anderson D. H., Ozaki S., Nealon M., Neitz J., Mullins R. F., Hageman G. S., Johnson L. V. Local cellular sources of apolipoprotein E in the human retina and retinal pigmented epithelium: implications for the process of drusen formation. Am. J. Ophthalmol. 2001; 131:767-781.
54. Curcio C. A., Johnson M., Huang J. D., Rudolf M. Aging, age-related macular degeneration, and the response-to-retention of apolipoprotein B-containing lipoproteins. Prog. Retin. Eye Res. 2009; 28:393-422.
55. Wang D., Zou L., Jin Q., Hou J., Ge G., Yang L. Human carboxylesterases: a comprehensive review. Acta Pharm. Sin. B. 2018; 8:699-712.
56. Edwards A. O., Klein M. L., Berselli C. B., Hejtmancik J. F., Rust K., Wirtz M. K., Weleber R. G., Acott T. S. Malattia leventinese: refinement of the genetic locus and phenotypic variability in autosomal dominant macular drusen. Am. J. Ophthalmol. 1998; 126:417-424.
57. Evans K., Gregory C. Y., Wijesuriya S. D., Kermani S., Jay M. R., Plant C., Bird A. C. Assessment of the phenotypic range seen in Doyne honeycomb retinal dystrophy. Arch. Ophthalmol. 1997; 115:904-910.
58. Storti F., Grimm C. Active Cholesterol Efflux in the Retina and Retinal Pigment Epithelium. Adv. Exp. Med. Biol. 2019; 1185:51-55.
59. Apte R. S. Targeting Tissue Lipids in Age-related Macular Degeneration. EBioMedicine. 2016; 5:26-27.
60. Biswas L., Zhou X., Dhillon B., Graham A., Shu X. Retinal pigment epithelium cholesterol efflux mediated by the 18ckDa translocator protein, TSPO, a potential target for treating age-related macular degeneration. Hum. Mol. Genet. 2017; 26:4327-4339.
61. Sene A., Khan A. A., Cox D., Nakamura R. E., Santeford A., Kim B. M., Sidhu R., Onken M. D., Harbour J. W., Hagbi-Levi S. Impaired cholesterol efflux in senescent macrophages promotes age-related macular degeneration. Cell Metab. 2013; 17:549-561.
62. Pool F. M., Kiel C., Serrano L., Luthert P. J. Repository of proposed pathways and protein-protein interaction networks in age-related macular degeneration. NPJ Aging Mech. Dis. 2020; 6:2.
63. Hotamisligil G. S. Inflammation and metabolic disorders. Nature. 2006; 444:860-867.
64. Ertunc M. E., Hotamisligil G. S. Lipid signaling and lipotoxicity in metaflammation: indications for metabolic disease pathogenesis and treatment. J. Lipid Res. 2016; 57:2099-2114.
65. Galloway C. A., Dalvi S., Hung S. S. C., MacDonald L. A., Latchney L. R., Wong R. C. B., Guymer R. H., Mackey D. A., Williams D. S., Chung M. M. Drusen in patient-derived hiPSC-RPE models of macular dystrophies. Proc. Natl. Acad. Sci. USA. 2017; 114:E8214-E8223.
66. Lakkaraju A., Finnemann S. C., Rodriguez-Boulan E. The lipofuscin fluorophore A2E perturbs cholesterol metabolism in retinal pigment epithelial cells. Proc. Natl. Acad. Sci. USA. 2007; 104:11026-11031.
67. Tabas I. Consequences of cellular cholesterol accumulation: basic concepts and physiological implications. J. Clin. Invest. 2002; 110:905-911.
68. Fu S., Fan J., Blanco J., Gimenez-Cassina A., Danial N. N., Watkins S. M., Hotamisligil G. S. Polysome profiling in liver identifies dynamic regulation of endoplasmic reticulum translatome by obesity and fasting. PLoS Genet. 2012; 8:e1002902.
69. Fu S., Yang L., Li P., Hofmann O., Dicker L., Hide W., Lin X., Watkins S. M., Ivanov A. R., Hotamisligil G. S. Aberrant lipid metabolism disrupts calcium homeostasis causing liver endoplasmic reticulum stress in obesity. Nature. 2011; 473:528-531.
70. Tumova J., Andel M., Trnka J. Excess of free fatty acids as a cause of metabolic dysfunction in skeletal muscle. Physiol. Res. 2016; 65:193-207.
71. Ban N., Lee T. J., Sene A., Choudhary M., Lekwuwa M., Dong Z., Santeford A., Lin J. B., Malek G., Ory D. S., Apte R. S. Impaired monocyte cholesterol clearance initiates age-related retinal degeneration and vision loss. JCI Insight. 2018; 3:3.
72. Li C. M., Chung B. H., Presley J. B., Malek G., Zhang X., Dashti N., Li L., Chen J., Bradley K., Kruth H. S., Curcio C. A. Lipoprotein-like particles and cholesteryl esters in human Bruch's membrane: initial characterization. Invest. Ophthalmol. Vis. Sci. 2005; 46:2576-2586.

1. A method of delaying the onset of, treating, preventing and/or curing a neurodegenerative disease in a subject in need thereof, comprising administering to the subject an effective amount of one or more agents which increase the expression or activity of CES1 in retinal pigment epithelium (RPE) cells.

2. The method of claim 1, wherein the one or more agents comprises one or more nucleic acids encoding CES1, or an active fragment or variant thereof.

3. The method of claim 2, wherein the one or more nucleic acids comprises one or more viral vectors which target the RPE.

4. The method of claim 3, wherein the one or more viral vectors comprise an adeno-associated viral (AAV) vector.

5. The method of claim 3 or claim 4, wherein the one or more viral vectors further comprise an RPE specific promoter.

6. The method of any of claims 1-5, wherein the one or more agents comprise one or more epidermal growth factor receptor (EGFR) signaling pathway agonists, or one or more nucleic acids encoding thereof.

7. The method of any of claims 1-6, wherein the one or more EGFR signaling pathway agonists comprise epidermal growth factor (EGF).

8. The method of any of claims 1-7, wherein the one or more agents comprises one or more inhibitors of SP1.

9. The method of any of claims 1-8, wherein the one or more agents regulate cholesterol efflux in RPE cells.

10. The method of any of claims 1-9, wherein the neurodegenerative disease is macular degeneration.

11. The method of any of claims 1-10, further comprising instructing the subject to ingest a low fat diet.

12. A method of delaying the onset of, treating, preventing and/or curing macular degeneration in a subject in need thereof, comprising administering to the subject an effective amount of one or more nucleic acids encoding a gene product which regulates cholesterol efflux.

13. The method of claim 12, wherein the nucleic acids are configured to express the gene product in retinal pigment epithelium (RPE) cells.

14. The method of claim 12 or 13, wherein the gene product comprises CES1 or an active fragment or variant thereof.

15. The method of any of claims 12-14, wherein the one or more nucleic acids comprises one or more viral vectors which target the RPE.
